(19) United States
(12) Patent Application Publication
(10) Pub. No.: US 2025/0049398 A1
(43) Pub. Date: Feb. 13, 2025

(54) DETECTION OF MEDICAL SCENARIO USING A HEARING INSTRUMENT

(71) Applicant: Starkey Laboratories, Inc., Eden Prairie, MN (US)

(72) Inventors: Edward John Cunningham, Savage, MN (US); Archelle Georgiou Feldshon, Wayzata, MN (US); Gregory John Haubrich, Champlin, MN (US); Brantly Sturgeon, Burnsville, MN (US); David Alan Fabry, Eden Prairie, MN (US)

(21) Appl. No.: 18/798,595

(22) Filed: Aug. 8, 2024

Related U.S. Application Data
(60) Provisional application No. 63/518,739, filed on Aug. 10, 2023.

(51) Int. Cl. A61B 5/00; A61B 5/0205; A61B 5/08; G06N 20/00; G16H 10/60

(52) U.S. Cl. CPC A61B 5/746; A61B 5/02055; A61B 5/0816; A61B 5/4803; A61B 5/6815; G06N 20/00; G16H 10/60

(57) ABSTRACT

An ear-wearable device comprising: a sensor configured to sense one or more physiological parameters of a user of the ear-wearable device; communications circuitry; and processing circuitry configured to: apply a machine learning (ML) model to the one or more sensed physiological parameters to determine whether the user is experiencing symptoms of a medical scenario, wherein the ML model is configured to be trained via a training set comprising a plurality of physiological parameter values and a corresponding plurality of symptoms of the medical scenario; and based on a determination that the user is experiencing symptoms of a medical scenario, cause the communications circuitry to transmit a notification indicating that the user is experiencing the symptoms of the medical scenario.

This application claims the benefit of U.S. provisional patent application 63/518,739 filed Aug. 10, 2023, and entitled “Detection of Medical Scenario Using a Hearing Instrument”, the entire content of which is incorporated by reference.

### TECHNICAL FIELD

This disclosure relates to ear-wearable devices.

### BACKGROUND

A user may use one or more ear-wearable devices for various purposes. For example, a user may use hearing instruments to enhance the user's ability to hear sound from a surrounding environment. In another example, a user may use hearing instruments to listen to media, such as music or television. Hearing instruments may include hearing aids, earbuds, headphones, earphones, personal sound amplifiers, cochlear implants, brainstem implants, osseointegrated hearing devices, or the like. A typical ear-wearable device includes one or more audio sources including microphone(s) and/or telecoil(s). The ear-wearable device may generate an audio signal representing a mix of sounds received by the one or more audio sources and produce a modified version of the received sound based on the audio signal. The modified version of the received sound may be different from the received sound.

### SUMMARY

This disclosure describes techniques for detecting, via one or more hearing instruments, whether a user is likely to experience a medical scenario. A user may ingest, intentionally or accidentally, one or more medical substances (e.g., a pharmaceutical substance, an illicit substance, alcohol) and consequently experience a medical scenario (e.g., a medical overdose, symptoms of medical substance addiction or withdrawal). A user may exhibit changes in physiological parameters as an effect of a medical substance that may correspond to an occurrence of a medical scenario or an imminent occurrence of a medical scenario. The physiological parameters may include, but are not limited to, respiration patterns of the user, speech patterns of the user, and/or choking or vomiting experienced by the user. An example hearing instrument described in this disclosure may sense physiological parameters from the user, determine a likelihood that the user is experiencing or will experience symptoms of a medical scenario, and transmit a notification or a request for medical aid to one or more parties (e.g., to a caretaker, to a medical care provider, to an emergency medical service (EMS) provider).

A system including hearing instrument(s) may store one or more machine learning (ML) models trained to determine a likelihood that a user is/will experience one or more medical scenarios (e.g., symptoms of medical substance addiction, symptoms of medical substance withdrawal, or a medical overdose) based on inputted physiological signal values. The system may receive the medicinal history of the user indicating the types and dosages of medical substances used by the user. Based on the medical history of the user, the system may select and apply different ML models to determine the likelihood of the one or more different scenarios. Different medical substances may have different effects on the user (e.g., may affect different physiological parameters). Each ML model may be configured to receive different physiological parameters as inputs to detect the likelihood of a corresponding medical scenario. Each ML model may correspond to a different medical substance than another ML model. ML models may be configured to distinguish when the user is likely to experience symptoms of a medical scenario from when the user may be experiencing another medical condition (e.g., stroke, cardiac arrest, hypoglycemia).

The systems, devices, and techniques described in this disclosure may provide one or more advantages over other medical scenario detection techniques. The systems, devices, and techniques described herein may determine the likelihood of a medical scenario based on physiological parameters sensed from multiple sensors, which may provide for a more accurate determination and may reduce the likelihood of false positive or false negative results. The systems, devices, and techniques described herein may determine the likelihood of a medical scenario before any outward symptoms of the medical scenario, which may increase a reaction time to provide medical aid to the user before the onset of the medical scenario.

In some examples, this disclosure describes a hearing instrument (e.g., an car-wearable device) comprising: a sensor configured to sense one or more physiological parameters of a user of the car-wearable device; communications circuitry; and processing circuitry configured to: apply a machine learning (ML) model to the one or more sensed physiological parameters to determine whether the user is experiencing symptoms of a medical scenario, wherein the ML model is configured to be trained via a training set comprising a plurality of physiological parameter values and a corresponding plurality of symptoms of the medical scenario; and based on a determination that the user is experiencing symptoms of the medical scenario, cause the communications circuitry to transmit a notification indicating that the user is experiencing the symptoms of the medical scenario.

In some examples, this disclosure describes a system comprising: an ear-wearable device configured to be worn in, on, or about an ear of a user, the ear-wearable device comprising: a sensor configured to sense one or more physiological parameters of the user; and memory; and a processing system configured to: retrieve, from car-wearable device, the one or more sensed physiological parameters; retrieve, from the memory, a machine learning (ML) model, wherein the ML model is configured to be trained via a training set comprising a plurality of physiological parameter values and a corresponding plurality of symptoms of a medical scenario; apply the ML mode to the one or more sensed physiological parameters to determine whether a likelihood that the user is experiencing or will experience the medical scenario; and based on a determination that the user is experiencing symptoms of the medical scenario, transmit a notification to a computing device indicating that the likelihood that the user is experiencing or will experience the medical scenario.

In some examples, this disclosure describes: a method comprising: sensing, via a sensor disposed within an ear-wearable device configured to be worn in, on, or about an car of a user, one or more physiological parameters of the user; applying, by processing circuitry of the car-wearable device, a machine learning (ML) model to the one or more physiological parameters to determine a likelihood that the user will experience a medical scenario; and transmitting, by the processing circuitry and via communications circuitry of the car-wearable device, a notification indicating the likelihood that the user will experience the medical scenario.

In some examples, this disclosure describes a computer-readable medium comprising instructions that, when executed, causes processing circuitry of an ear-wearable device to sense, via a sensor disposed within the car-wearable device, one or more physiological parameters of the user; apply a machine learning (ML) model to the one or more physiological parameters to determine a likelihood that the user will experience a medical scenario; and transmit, via communications circuitry of the car-wearable device, a notification indicating the likelihood that the user will experience the medical scenario

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 illustrates an example hearing instrument system, in accordance with one or more techniques of this disclosure.

FIG. 2 is a block diagram illustrating example components of an example hearing instrument of FIG. 1.

FIG. 3 is a block diagram illustrating an example external device of FIG. 1.

FIG. 4 is a conceptual diagram illustrating an example machine learning model stored in the example hearing instrument system of FIG. 1.

FIG. 5 is a block diagram illustrating an example process of training the example machine learning model of FIG. 4.

FIG. 6 is a block diagram illustrating an example process of applying the example machine learning model of FIG. 4.

FIG. 7 is a flow diagram illustrating an example process of determining a likelihood that a user is experiencing a medical scenario.

FIG. 8 is a flow diagram illustrating another example process of determining a likelihood that a user is experiencing a medical scenario.

### DETAILED DESCRIPTION

A user may use one or more hearing instruments to enhance, reduce, or modify sounds in an acoustic environment surrounding the user. Hearing instruments may be worn in, on, or about the ears of the user. Hearing instruments may include, but are not limited to, hearing aid, earbuds, headphones, earphones, personal sound amplifiers, cochlear implants, brainstem implants, osseointegrated hearing devices, or the like. In some examples, the user may wear a first hearing instrument around one ear and a second hearing instrument around another ear. Each of the first hearing instrument and the second hearing instrument may output a same sound or a different sound.

Hearing instruments may also include one or more sensors configured to sense physiological signals from the user. The one or more sensors may include, but are not limited to, motion sensors (e.g., accelerometers, gyroscopes, inertial measurement units (IMUs), a photoplethysmography (PPG) sensor, microphone(s), or oximeters. Hearing instruments may sense physiological signals from the user and determine the value and/or occurrence of one or more physiological parameters in the user. The physiological parameters may include, but are not limited to, a respiration pattern of the user (e.g., a delay between exhalation and inhalation by the user, an intra-breath spacing between temporally adjacent breaths), changes in the respiration pattern of the user, a speech pattern of the user (e.g., the presence or absence of slurred speech, changes in the speech rate), changes in the speech pattern of the user, the respiration rate of the user, changes in the respiration rate of the user, instances of vomiting experienced by the user, or instances of choking by the user.

Hearing instruments may determine the respiration rate and/or the respiration pattern of the user via motion-based sensing techniques (e.g., via an accelerometer, gyroscope, or IMU) and/or via PPG-based sensing techniques. Respiration by the user may induce variations in a motion sensor signal. During respiration, the body of the user moves according to the respiration rate of the user. For example, the chest of the user expands during inspiration phases and contracts during expiration phases. Additionally, the head of the user may move according to the respiration rate of the user. Variations in the motion sensor signal may correspond to inspiration and expiration phases in the respiration of the user. Hearing instruments may determine the respiration rate and the respiration pattern of the user based on the timing and magnitude of the variations in the motion sensor signal.

Respiration by the user may induce variations in a PPG signal in a variety of manners including, but are not limited to, Respiratory-Induced Intensity Variation (RIIV), Respiratory-Induced Amplitude Variation (RIAV), and/or Respiratory-Induced Frequency Variation (RIFV). RIIV may correspond to changes in a venous return of the user due to changes in intra-thoracic pressure throughout the respiratory cycle and may define a baseline (DC) modulation of the PPG signal. The RIAV may correspond to a reduction in a left ventricular stroke volume due to changes in intra-thoracic pressure causing a decreased pulse amplitude and vice versa. The RIFV may correspond to respiratory sinus arrythmia (RSA) during inspiration and expiration phases of the respiratory cycle. Hearing instruments may determine the respiration rate and respiration pattern of the patient based on the changes to, the presence of, or an absence of one or more of the variations in the PPG signal.

Hearing instruments may sense other physiological signals from the user via one or more sensors in hearing instruments. Hearing instruments may sense occurrences of choking or vomiting via microphone(s) in the hearing instruments. Hearing instruments may sense speech by the user via microphone(s) in the hearing instruments and may detect changes in the speech of the user (e.g., the presence of slurred speech). Hearing instruments may apply ML classification models to determine changes in the speech of the user or to determine occurrences of choking or vomiting by the user. Hearing instruments may sense body temperature of the user via a temperature sensor. Hearing instruments may sense a blood oxygen saturation level via an oximeter.

A hearing instrument system may input values for the physiological parameters into a machine learning (ML) model to determine a likelihood that the user is experiencing or will experience one or more medical scenarios (e.g., symptoms of a medical substance addiction, symptoms of a medical substance withdrawal, a medical overdose) resulting from a medical substance. The ML model may assign different weights to different physiological parameter to determine the likelihood. In some examples, the hearing instrument system receives user input indicating the medicinal history of the user. The medicinal history may indicate types and dosage of medical substances (e.g., pharmaceutical substances (e.g., prescription medications, over-the-counter medications), therapeutic substances, illicit substances, alcohol) ingested by the user. The hearing instrument system may select and apply specific ML models based on the medical substances ingested by the user, e.g., to monitor the likelihood of the one or more medical scenarios resulting from medical substances ingested or previously ingested by the user. Each ML models may be applied to determine a likelihood of a medical scenario, resulting from ingesting of a specific medical substance and may be configured to receive a different set of physiological parameters. Different medical substances may have different effects on different combinations of physiological parameters. Each ML model may be trained via physiological parameters affected by the medical substance corresponding to the ML model.

In some examples, the hearing instrument system may apply a ML model to determine a likelihood that the user is experiencing a medical overdose. In some examples, the hearing instrument system may apply a ML model to determine a likelihood of symptoms of medical substance addiction or symptoms of medical substance withdrawal. Determination of the likelihood of symptoms of substance addiction or substance withdrawal may be used by the user or a clinician to determine that the user is experiencing substance addiction before the user is likely to experience a medical overdose.

The hearing instrument systems, devices, and techniques described in this disclosure may provide one or more advantages over other medical scenario detection systems. The hearing instrument system described herein may determine the likelihood of medical scenario based on physiological signals sensed from multiple sensors, which may provide for a more accurate determination, may reduce the likelihood of false positive or false negative results, and/or may allow the use of physiological parameter values to verify a determination made by the hearing instrument system. The hearing instrument system may detect trends in the use of medical substances and the effects of the medical substances on the user to determine a likelihood of substance addiction, of substance withdrawal, or the likelihood of medical overdose before any outward symptoms are detected, which may increase the reaction time for the user to prepare for an advent of medical scenario (e.g., by contacting a medical care provider, by contacting an EMS provider).

Hearing instruments may be uniquely suited for this purpose. For example, because hearing instruments have microphones on opposite sides of the user's head, the hearing instruments are well-positioned to detect sounds made by the user and differentiate those sounds from external sounds. Additionally, hearing instruments with movement sensors are well-positioned to detect head and body movements that may not be detectable with other types of wearable devices, such as the movements associated with vomiting or agonal breathing. Hearing instruments are typically worn for prolonged periods of time, allowing collection of significant data. Furthermore, applying the ML model at the hearing instruments, as opposed to transmitting signals to other devices for analysis, may conserve battery power of the hearing instruments.

Applying the ML model at the hearing instruments may provide additional technological benefits compared to applying the ML model through other devices and/or system in communication with the hearing instruments. Application of the ML model at the hearing instruments may remove the need to transmit and/or receive signals to other devices and/or systems for the purpose of determining the likelihood of a medical scenario. The absence of the need to transmit and/or receive signals for this purpose may reduce power consumption by the hearing instruments and/or reduce the bandwidth required to transmit signals between the hearing instruments and other devices and/or systems. In some examples, applying the ML model at the hearing instruments reduces an amount of time between the detection of physiological signals and the determination of the likelihood of a medical scenario, e.g., by eliminating encryption, decryption, and/or transmission time needed to transmit signals between the hearing instruments and other devices and/or systems. In some examples, applying the ML model at the hearing instruments allow the techniques described herein to be performed even in scenarios with a loss of communications (e.g., loss of signal, loss of a data link) between the hearing instruments and other devices and/or systems. This may allow for the determination of the likelihood the patient is experiencing a medical scenario when the patient and the hearing instruments are situated in locations and/or situations where there is an inability to establish a communications channel between the hearing instruments and other devices and/or systems (e.g., on an aircraft, at a location with limited or no signal).

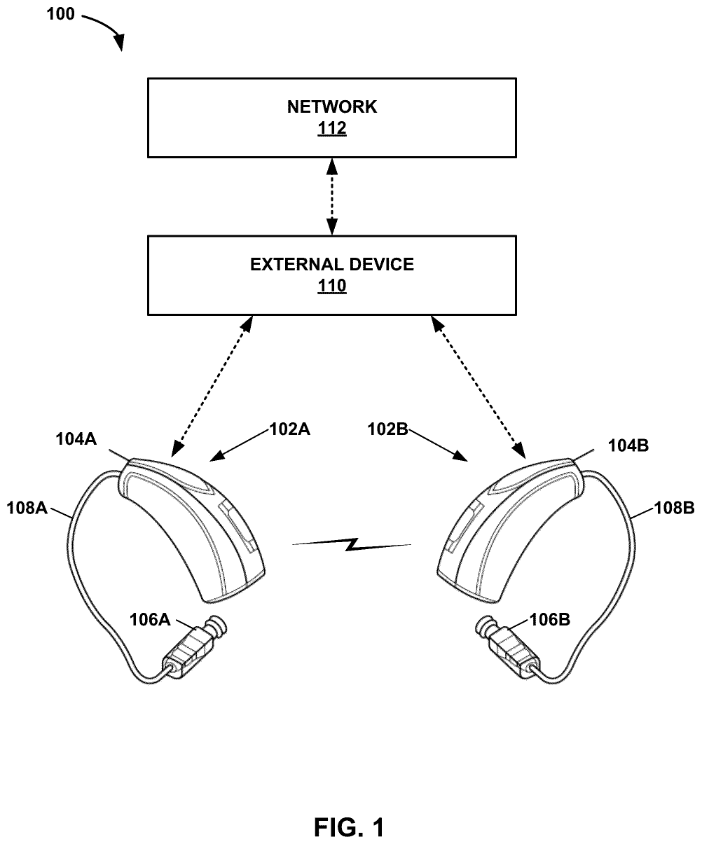

FIG. 1 illustrates an example hearing instrument system 100 (also referred to herein as “system 100”). System 100 includes a first hearing instrument 102A and a second hearing instrument 102B (collectively referred to as “hearing instruments 102”), external device 110, and network 112. Hearing instruments 102 may be alternatively referred to herein as “ear-wearable devices 102”. Hearing instruments 102 may be wearable concurrently in different cars of the same user. In some examples, the user may only wear one of hearing instruments 102 at a time. In the example of FIG. 1, hearing instruments 102 are shown as receiver-in-canal (RIC) style hearing aids. Thus, in the example of FIG. 1, first hearing instrument 102A includes a receiver-in-the-canal (RIC) unit 104A, a receiver unit 106A, and a communication cable 108A communicatively coupling RIC unit 104A and receiver unit 106A. Similarly, hearing instrument 102B includes a RIC unit 104B, a receiver unit 106B, and a communication cable 108B communicatively coupling RIC unit 104B and receiver unit 106B. RIC units 104A and 104B may be collectively referred to as “RIC units 104” or “processing units 104,” Receiver units 106A and 106B may be collectively referred to as “receiver units 106,” and communication cables 108A and 108B may be collectively referred to as “communication cables 108”. While the devices, systems, and methods of this disclosure are described primarily with reference to an RIC device (e.g., RIC units 104 of FIG. 1), the same techniques may be performed on other hearing instruments, computing systems and/or devices. For example, hearing instrument system 100 may include invisible-in-canal (IIC) devices, completely-in-canal (CIC) devices, in-the-canal (ITC) devices, in-the-car (ITE), behind-the-car (BTE) and other types of hearing instruments that reside within or about the user's ear. In instances where the techniques of this disclosure are implemented in IIC, CIC, ITC, or ITE devices, the functionality and components described in this disclosure with respect to RIC units 104 and receiver units 106 may be integrated into single enclosure.

It should be appreciated that hearing instruments 102 may form a Contralateral Routing of Signals (CROS) or a Bilateral Contralateral Routing of Signals (BiCROS) system wherein one of either hearing device 102A or hearing device 102B may primarily function to transmit audio from one ear to the opposite ear and, therefore, the audio transmitting device may lack a receiver unit and/or couple to the ear in a different manner than the receiving side device. In some examples, either hearing instrument 102A or hearing device 102B may function primarily to accept a user input or selection instead of transmitting, receiving, or processing audio input.

In the example of FIG. 1, first hearing instrument 102A may wirelessly communicate with second hearing instrument 102B and external device 110. In some examples, RIC units 104 include transmitters and receivers (e.g., transceivers) that support wireless communication between hearing instruments 102 and external device 110. In some examples, receiver units 106 include such transmitters and receivers (e.g., transceivers) that support wireless communication between hearing instruments 102 and external device 110. External device 110 may include a personal computer, a laptop, a tablet, a smartphone, a smartwatch, a cloud computer, a mesh network node, an internet gateway device, or the like.

Each of hearing instruments 102 may receive input audio signals from an environment surrounding the user, apply a processing mode to the input audio signals to generate output audio signals, and output a sound to the user based on the output audio signals. For example, each of RIC units 104 may receive sound from the environment in the form of input audio signals and generate the output audio signals based on the input audio signals and the processing mode. Each of receiver units 106 may then output the sound based on the output audio signals.

Each of hearing instruments 102 may sense physiological signals from the user via sensor(s) within or coupled to hearing instruments 102. The physiological signals may include, but are not limited, motion sensor signals, PPG signals, electroencephalogram (EEG) signals, electrocardiogram (ECG) signals, sensed temperature values, sensed blood oximetry values, audio signals or the like. Hearing instruments 102 may convert the sensed physiological signals to physiological parameter values. Physiological parameter values may include, but are not limited to, a respiration rate of the user, change in the respiration rate of the user, respiration pattern of the user, changes in the respiration pattern of the user, speech pattern of the user, changes in the speech pattern of the user, choking by the user, vomiting by the user, body temperature of the user, heart rate of the user, blood oxygen saturation of the user, or the like.

System 100 may determine a likelihood that the user is experiencing or will experience a medical scenario (e.g., medical substance addiction symptoms, medical substance withdrawal symptoms, or medical overdose) based on the physiological parameter values. A medical substance may include, but are not limited to, a pharmaceutical substance, a therapeutic substance, an illicit substance, alcohol, tobacco, or any other substances that may be ingested by the user and which may alter physiological function of the user. Although this disclosure primarily describes medical substances that are orally ingested by the user, the techniques described herein may be applied to medical substances ingested or otherwise inserted into the body of the user via any other means including, but are not limited to, nasally, via an injection (e.g., intravenous injection, intramuscular injection, subcutaneous injection), via an application on skin of the user, via a suppository, or the like.

System 100 (e.g., one of hearing instruments 102 of system 100, external device 110) may select a ML model stored in system 100 and input physiological parameter values into the ML model to output a likelihood that the user is experiencing or will experience a medical scenario. The ML model may be trained with a data set including prior physiological parameter values (e.g., from the user, from one or more other individuals) and a corresponding presence and/or severity of one or more symptoms or physiological effects of the medical condition. In some examples, the ML model is configured to determine the likelihood of the medical scenario based on a respiration pattern, a speech pattern, and an occurrence of choking or vomiting by the user. The ML model may assign a different weight or a different confidence level for each inputted physiological parameter within the ML model. The ML model may weigh values for each inputted physiological parameter based on the assigned weight or confidence level for the inputted physiological parameter. For example, the ML model may assign greater weight to an occurrence of choking or vomiting by the user and may assign a lower weight to a speech pattern of the user. The ML model may determine the weights or confidence levels for the inputted physiological parameters for the different inputted physiological parameters as a part of the training of the ML model.

System 100 may store a plurality of ML models. Each ML model may be configured to determine a likelihood of a different medical scenario (e.g., symptoms of substance addiction, symptoms of substance withdrawal, medical overdose, or an imminent medical overdose) for one or more medical substances. Different ML models may correspond to different medical substances. Different ML models may be configured to accept different combinations of physiological parameter(s) e.g., due to different effects of different medical substances on physiological parameters. System 100 may receive user input indicating medicinal history of the user. The medicinal history may indicate a type and dosage of one or more medical substances currently ingested by the user or ingested by the user within a threshold period of time (e.g., within the past month, within the past six months, within the past year). The medicinal history may also indicate a frequency of use and a last use time for each medical substance. System 100 may select and apply one or more ML models of the plurality of ML models to determine the likelihood of a medical scenario based on the medicinal history of the user (e.g., based on the types and dosages of the medical substances ingested by the user).

System 100 may verify the determination outputted by one or more ML models. System 100 may receive physiological signal data and may determine whether the received data is indicative of symptoms consistent with medical overdose. In some examples, system 100 determines whether the received physiological data, alone or in conjunction with the input physiological parameter values, is consistent with medical overdose or is inconsistent with medical overdose but consistent with another medical condition. System 100 may determine a period of time between the user ingesting a substance and the start of the physiological effects indicated in the physiological parameters (e.g., changes to the respiration rate, respiration pattern, speech pattern, heart rate, body temperature, blood pressure, balance, gait, the blood oxygen saturation level of user, the start of choking by the user, or the start of vomiting by the user). System 100 may verify the outputted determination from the ML model based on a determination that the period of time is consistent with an onset of action for a medical substance.

In some examples, a user may ingest multiple medical substances simultaneously or within rapid succession of each other such that the user simultaneously experiences the effects of two or more medical substances. System 100 may apply additional ML models based on additional medical substances ingested by the user to determine the likelihood that the user will experience a medical overdose for each specific medical substance and for two or more medical substances acting in concert.

System 100 may compare a determined likelihood against a threshold likelihood stored in system 100. The threshold likelihood may include a threshold likelihood of substance addiction, a threshold likelihood of substance withdrawal, a threshold likelihood that the user is experiencing a medical overdose, or a threshold likelihood that the user will experience a medical overdose. The threshold likelihood may represent a minimum likelihood of a medical scenario where the user may require monitoring of medical care. System 100 may determine that the determined likelihood satisfies the threshold likelihood (e.g., that the determined likelihood is greater than or equal to the threshold likelihood) and output a notification in response to the determination. The threshold likelihood may be entered into system 100 by the user or by a clinician.

Hearing instruments 102 may transmit the notification to external device 110. Hearing instruments 102 and external device 110 may transmit the notification to one or more computing devices, systems, or cloud computing environments in communication with hearing instruments 102 and/or external device 110. The notification may be transmitted to external device 110 associated with the user (e.g., a smartwatch, a smartphone, a laptop, and/or a laptop associated with the user), to a computing device or system associated within one or more persons associated within the user in network 112 (e.g., a family member, a caretaker), or a computing device or system associated with a medical care provider or with an EMS provider connected to network 112. The notification may include a likelihood that the user is experiencing or will experience a medical overdose, a type of medical overdose, and/or a log of the types and amounts of substances ingested by the user. In some examples, the notification displays locations of the user and nearby medical care locations and/or EMS locations, e.g., to facilitate the delivery of medical aid to the user.

System 100 may output the notification to different third parties based on a determined severity of the medical scenario. For example, system 100 may output the notification to family members and caretakers for a less severe medical scenario and may output the notification to medical care providers or to EMS providers in response to a more severe medical scenario. System 100 may determine a severity of a medical scenario based on a determination that the determined likelihood satisfies a threshold severity likelihood (e.g., is less than or equal to 25%, is greater than or equal to 50%, is greater than or equal to 80%). The threshold severity likelihood may be determined by the user or by a clinician.

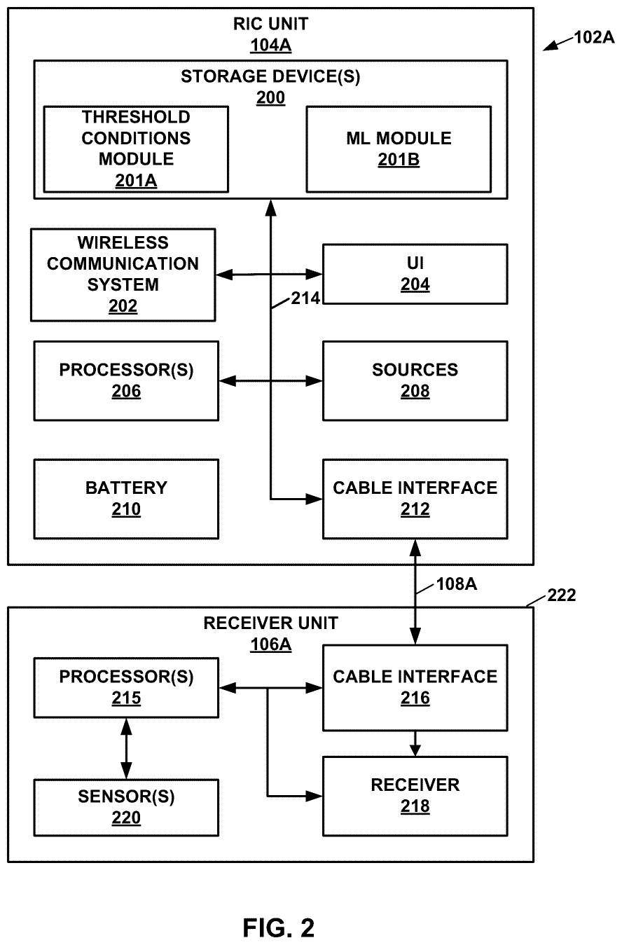

FIG. 2 is a block diagram illustrating example components of an example hearing instrument of FIG. 1. As illustrated in FIG. 2, hearing instrument 102A includes RIC unit 104A and receiver unit 106A configured according to one or more techniques of this disclosure. Hearing instrument 102B may include similar components to those shown in FIG. 2. In another example, other hearing instruments 102 include the components described herein in a single device (e.g., in a single IIC or CIC device).

In the example of FIG. 2, RIC unit 104A includes one or more storage device(s) 200, a wireless communication system 202, user interface (UI) 204, one or more processor(s) 206, one or more sources 208, a battery 210, a cable interface 212, and communication channels 214. Communication channels 214 provide communication between storage device(s) 200, wireless communication system 202, processor(s) 206, microphones 208, and cable interface 212. Storage devices 200, wireless communication system 202, processors 206, microphones 208, cable interface 212, and communication channels 214 may draw electrical power from battery 210, e.g., via appropriate power transmission circuitry. In other examples, RIC unit 104A may include more, fewer, or different components. For instance, RIC unit 104A may include a wired communication system instead of a wireless communication system and RIC unit 104A and RIC unit 104B may be connected via the wired communication system.

Furthermore, in the example of FIG. 2, receiver unit 106A includes one or more processors 215, a cable interface 216, a receiver 218, and one or more sensors 220. In other examples, receiver unit 106A may include more, fewer, or different components. For instance, in some examples, receiver unit 106A does not include sensors 220 or receiver unit 106A may include an acoustic valve that provides occlusion when desired. In some examples, receiver unit 106A has a housing 222 that may contain some or all components of receiver unit 106A (e.g., processors 215, cable interface 216, receiver 218, and sensors 220). Housing 222 may be a standard shape or may be customized to fit a specific user's ear.

Storage device(s) 200 of RIC unit 104A include devices configured to store data. Such data may include computer-executable instructions, such as software instructions or firmware instructions. Storage device(s) 200 may include volatile memory and may therefore not retain stored contents if powered off. Examples of volatile memories may include random access memories (RAM), dynamic random access memories (DRAM), static random access memories (SRAM), and other forms of volatile memories known in the art. Storage device(s) 200 may further be configured for long-term storage of information as non-volatile memory space and retain information after power on/off cycles. Examples of non-volatile memory configurations may include flash memories, or forms of electrically programmable memories (EPROM) or electrically erasable and programmable (EEPROM) memories. Hearing instrument 102A may be configured to transmit the stored information from storage device(s) 200 to external device 110, network 112, and/or one or more other computing devices, computing systems, and/or cloud computing environments.

Storage device(s) 200 may define one or more modules (e.g., threshold conditions module 201A, machine learning (ML) module 201B), collectively referred to as “modules 201,” each of modules 201 being configured to store different types of information. Threshold conditions module 201A may store threshold likelihood values for a plurality of medical overdoses. The threshold likelihood values may correspond to threshold likelihoods that the user would experience symptoms of substance addiction, threshold likelihoods of that the user would experience symptoms of substance withdrawal, threshold likelihoods that the user is experiencing a medical overdose, or threshold likelihoods that the user will experience a medical overdose for one or more types of medical substances. ML module 201B may store a plurality of ML models. Each ML model may be a Decision Tree model or may be implemented as an artificial neural network (ANN). The ANN may be a fully connected model that includes one or more hidden layers. The ANN may use a sigmoid activation function, rectified linear unit (ReLU) activation function, or another activation function. In other examples, the ML models may include a support vector machine (SVM), or other type of ML model. Each ML model may be configured to determine a likelihood of one or more occurrences (e.g., that the user would experience symptoms of substance abuse, that the user would experience symptoms of substance withdrawal, that the user would experience medical overdose, that the user will imminently experience a medical overdose). Each ML model may correspond to a specific medical substance. For example, a first ML model may correspond to opioids and a second ML model may correspond to alcohol.

Wireless communication system 202 may enable RIC unit 104A to send data to and receive data from one or more other computing devices, e.g., external device 110, hearing instrument 102B. Wireless communication system 202 may use various types of wireless technology to communicate. For instance, wireless communication system 202 may use Bluetooth, Bluetooth LE, 3G, 4G, 4G LTE, 5G, ZigBee, WiFi, Near-Field Magnetic Induction (NFMI), or another communication technology. In other examples, RIC unit 104A includes a wired communication system that enables RIC unit 104A to communicate with one or more other devices, such as hearing instrument 102B, via a communication cable, such as a Universal Serial Bus (USB) cable or a Lightning™ cable.

Sources 208 include one or more components configured to convert an input (e.g., sound, electromagnetic energy) into electrical signals. In other words, sources 208 may generate one or more input audio signals. Sources 208 may include, but are not limited to, microphones and telecoils. While sources 208 are described primarily with reference to microphones and telecoils herein, it may be appreciated that the techniques may be applied to input audio signals from one or more other sources 208. In some examples, sources 208 are included in receiver unit 106A instead of RIC unit 104A. In some examples, one or more of sources 208 are included in RIC unit 104A and one or more of sources 208 are included in receiver unit 106A.

Sources 208 may include microphones configured to convert sound into electrical signals. In some examples, sources 208 include a front microphone and a rear microphone. The front microphone may be located closer to the front (i.e., ventral side) of the user. The rear microphone may be located closer to the rear (i.e., dorsal side) of the user. One or more of sources 208 are omnidirectional microphones, directional microphones, or another type of microphones. Sources 208 may include one or more telecoils. The telecoils may detect wireless signals modulated to carry audio signals. For example, the telecoils may detect electromagnetic energy and detect an audio signal carried by the energy. In some examples, one or more of sources 208 may be one or more external microphones or telecoils operatively connected to hearing devices 102 using an electromagnetic audio or data transmission scheme, e.g., Bluetooth, Bluetooth LE, 900 MHz, 2.4GHz, FM, infrared, 3G, 4G, 4G LTE, 5G, ZigBee, WiFi, Near-Field Magnetic Induction (NFMI) and the like.

Processor(s) 206 (also referred to as “processing system 206”) include circuitry configured to process information. RIC unit 104A may include various types of processor(s) 206. For example, RIC unit 104A may include one or more microprocessors, digital signal processors, microcontroller units, and other types of circuitries for processing information. In some examples, one or more of processor(s) 206 may retrieve and execute instructions stored in one or more of storage device(s) 200. The instructions may include software instructions, firmware instructions, or another type of computer-executed instructions. In accordance with the techniques of this disclosure, processor(s) 206 may perform processes for retrieving sensed physiological parameter values from sensor(s) 220 of hearing instrument 102A, input the sensed physiological parameter values into a ML model retrieved from ML module 201B of storage device(s) 200, and apply the ML model to determine a likelihood that the user may experience one or more medical scenarios (e.g., symptoms of substance addiction, symptoms of substance withdrawal, and/or an occurrence or imminent occurrence of a medical overdose). In different examples of this disclosure, processor(s) 206 may perform such processes fully or partly by executing such instructions, or fully or partly in hardware, or a combination of hardware and execution of instructions.

Processor(s) 206 may retrieve and execute instructions from storage device(s) 200 (e.g., from ML module 201B) corresponding to a ML model to apply the ML model and determine the likelihood that the user may experience the one or more medical scenarios. Processor(s) 206 may select and apply different ML models based on different medical substances ingested by the user and/or based on different types of medical scenarios. For example, processor(s) 206 may select and apply a first ML model to determine a likelihood that the user will experience a medical overdose and select and apply a second ML model different from the first ML model to determine a likelihood that the user will experience symptoms of medical substance addiction.

Each ML model may be trained, e.g., by external device 110 and/or one or more computing devices, systems, and/or cloud computing environments connected to network 112, using a training set including past physiological parameter values and corresponding symptoms, severity of symptoms, and/or type of medical scenario. The training set may include data from the user and/or one or more other individuals who are similar to the user (e.g., also using hearing instruments 102 or similar hearing instruments, have similar hearing impairment and/or other conditions, ingesting or having ingested similar medical substances). When applied by processor(s) 206, each ML model may determine, based on sensed physiological parameters from the user, a likelihood of a medical scenario (e.g., a likelihood that the user is experiencing symptoms of medical substance addiction, of medical substance withdrawal, or of a medical overdose). The likelihood may be represented as a percentage, as a ratio, as a score, or in terms of a label (e.g., “very low likelihood,” “low likelihood,” “moderate likelihood,” “high likelihood,” “very high likelihood”).

UI 204 may be configured to transmit notifications to the user and/or receive user input and/or user selection. UI 204 may include, but are not limited to, lights, buttons, dials, switches, microphones, a haptic feedback component, or the like. UI 204 may be configured to receive tactile, gestural (e.g., movement of a head, or a limb of the user), visual and/or auditory feedback from the user indicating user input. UI 204 may then convert the received feedback into electrical signals and transmit the electrical signals to other components within hearing instrument 102A via communications channels 214. UI 204 may also receive instructions to transmit a notification to the user via communications channels 214 and output a visual, auditory, and/or tactile feedback to the patient. UI 204 may be in communication with receiver unit 106A and may receive feedback from and/or transmit notifications to the user via one or more components of receiver unit 106A, e.g., receiver 218.

In the example of FIG. 2, cable interface 212 is configured to connect RIC unit 104A to communication cable 108A. Communication cable 108A enables communication between RIC unit 104A and receiver unit 106B. Cable interface 212 may include a set of pins configured to connect to wires of communication cable 108A. In some examples, cable interface 212 includes circuitry configured to convert signals received from communication channels 214 to signals suitable for transmission on communication cable 108A. Cable interface 212 may also include circuitry configured to convert signals received from communication cable 108A into signals suitable for use by components in RIC unit 104A, such as processors 206. In some examples, cable interface 212 is integrated into one or more of processor(s) 206. Communication cable 108 may also enable RIC unit 104A to deliver electrical energy to receiver unit 106.

In some examples, communication cable 108A includes a plurality of wires. The wires may include a Vdd wire and a ground wire configured to provide electrical energy to receiver unit 106A. The wires may also include a serial data wire that carries data signals and a clock wire that carries a clock signal. For instance, the wires may implement an Inter-Integrated Circuit (I²C bus). Furthermore, in some examples, the wires of communication cable 108A may include receiver signal wires configured to carry electrical signals (e.g., output audio signals) that may be converted by receiver 218 into sound.

In the example of FIG. 2, cable interface 216 of receiver unit 106A is configured to connect receiver unit 106A to communication cable 108A. For instance, cable interface 216 may include a set of pins configured to connect to wires of communication cable 108A. In some examples, cable interface 216 includes circuitry that converts signals received from communication cable 108A to signals suitable for use by processors 215, receiver 218, and/or other components of receiver unit 106A. In some examples, cable interface 216 includes circuitry that converts signals generated within receiver unit 106A (e.g., by processors 215, sensors 220, or other components of receiver unit 106A) into signals suitable for transmission on communication cable 108A.

Receiver unit 106A may include various types of sensor(s) 220. Sensor(s) 220 may include accelerometers, gyroscopes, IMUs, heartrate monitors, PPG sensors, temperature sensors, microphones, oximeters, and the like. In some examples, at least some of the sensors may be disposed within RIC unit 104A. Like processor(s) 206, processor(s) 215 include circuitry configured to process information. For example, processor(s) 215 may include one or more microprocessors, digital signal processors, microcontroller units, and other types of circuitry for processing information. In some examples, processor(s) 215 may process signals from sensor(s) 220. In some examples, processor(s) 215 process the signals from sensor(s) 220 for transmission to RIC unit 104A. Signals from sensor(s) 220 may be used for various purposes, such as for determining a likelihood that the user is experiencing one or more of the medical scenarios as previously described herein.

In some examples, processor(s) 215 may apply ML classification model(s) to determine the speech pattern of the user, vomiting, and/or choking by the user based on audio signals recorded by hearing instruments 102. Processor(s) 215 may apply a first ML classification model to determine the speech pattern and/or changes in the speech pattern (e.g., slurred speech, garbled speech, changes in the speaking rate) of the user. The first ML classification model may be trained via training data including audio signals from the user and/or one or more other individuals and corresponding labels indicating the speech rate and the clarity of the speech. Processor(s) 215 may input audio signals sensed from sensor(s) 220 into the first ML classification model to determine a speech rate and a speech clarity of the user.

Processor(s) 215 may apply a second ML classification model to detect vomiting and/or choking by the user based on an input audio signal. The second ML classification model may be trained via training data including audio signals from the user and/or one or more other individuals and corresponding labels indicating the presence of vomiting or choking in the audio signals. Processor(s) 215 may input audio signals sensed from sensor(s) 220 into second ML classification model to determine the presence and frequency of vomiting or choking by the user.

Processor(s) 206 and/or processor(s) 215 may receive (e.g., from sensor(s) 220) physiological signals from the user. The physiological signals may include, but are not limited to, motion sensor signals, PPG signals, heart rate signals, blood oximetry signals, input audio signals, or the like. Processor(s) 206 and/or processor(s) 215 may convert the received physiological signals into physiological parameter values. Physiological parameters may include respiration rate, respiration patterns, speech patterns, occurrences of vomiting, occurrences of choking, body temperature, blood oxygen saturation, heart rate, balance, gait, or the like. Respiration patterns may include a time period between exhalation and inhalation by the user or an intra-breath spacing between temporally adjacent breaths by the user. Processor(s) 206 and/or process(s) 215 may store the physiological parameter values in storage device(s) 200.

Processor(s) 206 and/or processor(s) 215 may retrieve and execute instructions for one or more ML models from storage device(s) 200 to apply the one or more ML models to the physiological parameter values to determine a likelihood that the user is experiencing or will experience one or more medical scenarios. Processor(s) 206 and/or processor(s) 215 may input one or more physiological parameter values into a ML model and receive a determined likelihood of a medical scenario from the ML model. Processor(s) 206 and/or processor(s) 215 may select and input different physiological parameters into different ML models based on the physiological parameters each ML model is configured to receive. In some examples, processor(s) 206 and/or processor(s) 215 may input a respiration pattern, a speech pattern, and instances of choking or vomiting of the user into a ML model to determine a likelihood that the user is/will experience a medical overdose. Processor(s) 206 and/or processor(s) 215 may constantly apply ML models to determine the likelihoods of the occurrence at different times, may apply ML models in response to user input, or may apply ML models in response to detection of an action by the user consistent with ingestion of a medical substance (e.g., movement of the head of the user that is consistent with swallowing of an object).

Processor(s) 206 and/or processor(s) 215 may receive (e.g., via wireless communication system 202, UI 204, and/or sensor(s) 220) user input indicating medicinal history of the user. Processor(s) 206 and/or processors (2) 215 may select ML models from the plurality of ML models stored in storage device(s) 200 based on the types and/or dosage of medical substances ingested by the user, as indicated in the user input. Each ML model may correspond to a different medical substance or a different combination of medical substances and may be trained to determine a likelihood of an medical scenario resulting from the different medical substance or the different combination of medical substances. Processor(s) 206 and/or processor(s) 215 may adjust, based on subsequent user input, changes in the types and/or dosages of medical substances ingested by the user. Processor(s) 206 and/or processor(s) 215 may adjust the selection of ML models accordingly.

In some examples, processor(s) 206 and/or processor(s) 215 may determine at least a portion of the medicinal history (e.g., a substance-use history) of the user based on the sensed physiological signals (e.g., from sensor(s) 220). For example, processor(s) 206 and/or processor(s) 215 may determine medication events or occurrences (e.g., when the user experiences pharmaceutical effects from an ingested substance) from the sensed physiological signals. Processor(s) 206 and/or processor(s) 215 may generate a substance-use history for the patient for each ingested substance based on the determined medication events.

Processor(s) 206 and/or processor(s) 215 may determine, based on the substance-use history and/or the received medicinal history, whether the user is ingesting medicinal substances in compliance with a medical prescription or is abusing one or more medicinal substances. Processor(s) 206 and/or processor(s) 215 may determine that the user is abusing a medical substance based on a determination that the user has experienced a medication event consistent with the effects of the medical substance after the expiration of a prescription and/or at a magnitude or rate in excess of an instructed dosage or dosage-rate for the prescription. Similarly, processor(s) 206 and/or processor(s) 215 may determine that the user is compliant with a medical prescription by determining that the user is experiencing the medication event within a time period for a prescription and at a dose and dose-rate as indicated in the prescription. Processor(s) 206 and/or processor(s) 215 may receive prescription information (e.g., medication type, duration, dosage, dosage-rate) as a part of the medicinal history inputted or may retrieve the prescription information from a computing device, system, or cloud computing environment in communication with wireless communication system 202.

Processor(s) 206 and/or processor(s) 215 may compare the determined likelihood of an occurrence outputted by a ML model against a threshold likelihood stored in threshold conditions module 201A. Processor(s) 206 and/or processor(s) 215 may determine whether the determined likelihood of an occurrence satisfies a threshold likelihood of occurrence. The threshold likelihood may correspond to a same type of medical scenario and same medical substance(s) as the ML model. Threshold likelihoods may be entered into hearing instrument 102A by the user and/or by a clinician, e.g., via external device 110.

Processor(s) 206 and/or processor(s) 215 may verify a determined likelihood of a medical scenario. In some examples, processor(s) 206 and/or processor(s) 215 receives sensor data (e.g., from sensor(s) 220, from computing devices and/or systems in communication with wireless communication system 202) and determines whether the received sensor data is consistent with the determined likelihood of the occurrence. For example, processor(s) 206 and/or processor(s) 215 may determine that the received sensor data is consistent with a symptom of the type of medical scenario, e.g., whether the sensor data shows physiological parameters or behavior consistent with a symptom of medical overdose. Processor(s) 206 and/or processor(s) 215 may determine whether the physiological parameters corresponding to the received sensor data are consistent with any other medical conditions the user may experience. In some examples, processor(s) 206 and/or processor(s) 215 may determine (e.g., via input audio signals and/or motion sensor signals), a time period between ingestion of the medical substance and a time when processor(s) 206 and/or processor(s) 215 determines that the likelihood of the medical scenario satisfies a threshold likelihood stored in storage device(s) 200. Processor(s) 206 and/or processor(s) 215 may verify the determined likelihood based on a determination that the time period is consistent with an onset of action period of the medical substance.

Processor(s) 206 and/or processor(s) 215 may cause wireless communication system 202 to output a notification to one or more computing devices, computing systems, and/or cloud computing environments in communication with wireless communication system 202 via network 112. The one or more computing devices, computing systems, and/or cloud computing environments may correspond to family members of the user, caretaker(s) of the user, medical care providers, or EMS providers. The notification may include likelihoods of one or more medical scenarios and the corresponding medical substances. For example, the notification may include a likelihood the user is/will experience symptoms of addiction, symptoms of withdrawal, and/or a medical overdose resulting from a medical substance (e.g., from an opioid, from alcohol). The notification may indicate types and dosages of medical substances ingested by the patient. In some examples, the notification may include a location of hearing instrument 102A (e.g., as retrieved from external device 110 or from network 112). The location of hearing instrument 102A may facilitate delivery of medical aid to the user.

Processor(s) 206, 215, may cause wireless communication system 202 to output the notification to different parties based on a severity of the medical scenario. The severity may be based on the likelihood of the medical scenario (e.g., whether the determined likelihood satisfies one or more threshold severity likelihoods) or may be based on the type of medical scenario and/or ML model. For example, processor(s) 206, 215 may apply different ML models to determine the likelihood of a medical scenario at different levels of severity. Processor(s) 206, 215 may cause wireless communication system 202 to output the notification to the user, to family members, or to caretakers for a less severe medical scenario. Processor(s) 206, 215 may cause wireless communication system 202 to output the notification to medical care providers or to EMS providers for a more severe medical scenario.

Processor(s) 206 and/or processor(s) 215 may cause wireless communication system 202 to output a notification to external device 110, e.g., to alert the user to the likelihood of an medical scenario. The notification may indicate the likelihood that the user is/will experience the medical scenario, the type of the medical scenario, the severity of the medical scenario, and/or the medical substance(s) likely to cause the medical scenario. The notification may include the location of hearing instrument 102A and locations of nearby health care providers or EMS providers, e.g., to facilitate the user in seeking medical aid.

Processor(s) 206, 215 may transmit a notification to one or more vehicles or other machinery, e.g., to inhibit the user from operating the vehicle or other machinery while the user is likely to be impaired. For example, processor(s) 206, 215 may determine a likelihood that the user is intoxicated based on sensed physiological parameters (e.g., a balance, speech pattern, a gait of the user). Processor(s) 206, 215 may then transmit the notification to the vehicle or to network 112 in communication with the vehicle. Based on the notification, network 112 and/or the vehicle may engage a driving interlock of the vehicle based on a determination that the user is in the proximity of the vehicle (e.g., via location information of hearing instruments 102 or of external device 110).

In other examples, hearing instruments 102 (FIG. 1) may be implemented as a BTE device in which components shown in receiver unit 106A are included in a housing having similar functions to RIC unit 104A secured behind the car of the user and a sound tube extends from receiver 218 into the user's ear. The sound tube may comprise an air-filled tube that channels sound into the user's ear. In such examples, cable interface 212, cable interface 216, and processors 215 may be omitted. Furthermore, in such examples, receiver 218 may be integrated into the housing. In some examples, sensors 220 may be integrated into the RIC unit.

In some examples, hearing instruments 102 includes an car-wearable device 102A comprising: a sensor 220 configured to sense one or more physiological parameters of a user of the car-wearable device 102A, communications circuitry (e.g., wireless communication system 202), and processing circuitry (e.g., processor(s) 206, 215). The processing circuitry may be configured to apply a ML model to the one or more sensed physiological parameters to determine whether the user is experiencing symptoms of a medical overdose, wherein the ML model is configured to be trained via a training set comprising a plurality of physiological parameter values and a corresponding plurality of symptoms of the medical overdose; and based on a determination that the user is experiencing symptoms of a medical overdose, cause the communications circuitry to transmit a notification indicating that the user is experiencing the symptoms of the medical overdose.

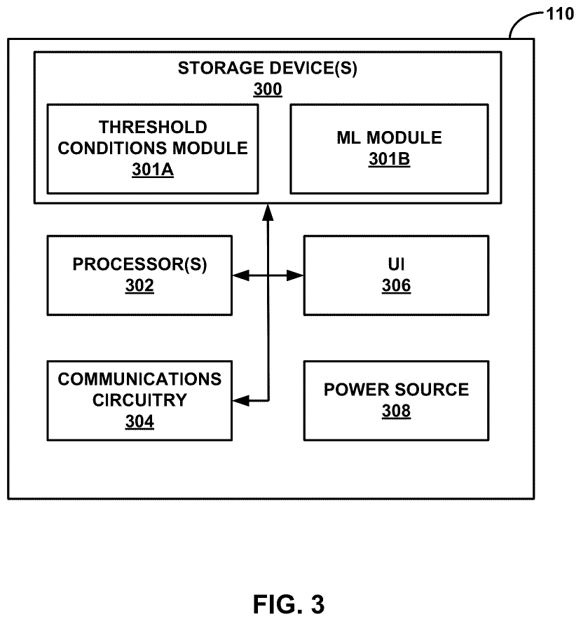

FIG. 3 is a block diagram illustrating an example external device 110 of FIG. 1. As illustrated in FIG. 3, external device 110 may include storage device(s) 300, processor(s) 302, communications circuitry 304, user interface (UI) 306, and power source 308. In other examples, external device 110 may include more or fewer components than the example external device 100 illustrated in FIG. 3. In some examples, hearing instruments 102 may communicate directly with network 112 and the components and functions illustrated in FIG. 3 may be implemented by network 112 and/or one or more other computing devices, computing systems, and/or cloud computing environments connected to network 112. External device 100 may include, but are not limited to, a personal computing device of the user (e.g., a cellphone, a smartphone, a smartwatch, a tablet, a laptop, a personal computer), an external programmer for hearing instruments 102, a charger for hearing instruments 102, or other accessories configured to provide a communications link between hearing instruments 102 and network 112 (e.g., a health monitoring network).

Storage device(s) 300 of external device 110 include devices configured to store data. Such data may include computer-executable instructions, such as software instructions or firmware instructions. Storage device(s) 300 may include volatile memory and may therefore not retain stored contents if powered off. Examples of volatile memories may include random access memories (RAM), dynamic random access memories (DRAM), static random access memories (SRAM), and other forms of volatile memories known in the art. Storage device(s) 300 may further be configured for long-term storage of information as non-volatile memory space and retain information after power on/off cycles. Examples of non-volatile memory configurations may include flash memories, or forms of electrically programmable memories (EPROM) or electrically erasable and programmable (EEPROM) memories.

Storage device(s) 300 may store data corresponding to threshold conditions (e.g., threshold likelihoods) for each of a plurality of possible scenarios a user may experience. Possible scenarios may include, but are not limited to, a threshold likelihood that the user is or will experience symptoms of medical substance addiction, a threshold likelihood that the user is or will experience symptoms of medical substance withdrawal, a threshold likelihood that the user is experiencing a medical overdose, or a threshold likelihood that the user will experience a medical overdose. Storage device(s) 300 may store data corresponding to threshold conditions for each possible scenario for each of a plurality of medical substances. Storage device(s) 300 may store the data corresponding to threshold conditions in threshold conditions module 301A. External device 110 may receive, via communications circuitry 304 or via UI 306, user input indicating the medicinal history of the user. External device 100 may then transmit data corresponding to threshold conditions for medical substances included in the medicinal history to hearing instruments 102. Hearing instruments 102 may determine whether the likelihood that the user is experiencing a specific scenario satisfies a corresponding threshold condition transmitted by external device 110 to hearing instruments 102.

Storage device(s) 300 may store data corresponding to one or more ML classification models and/or to one or more ML models, e.g., in ML module 301B. Each ML classification model may be configured to determine physiological parameter values based on sensed physiological signals. Each ML model may be configured to determine a likelihood that the user is experiencing a medical scenario based on physiological parameter values. Each ML model may be trained to determine a likelihood that the user is experiencing or will experience a specific scenario and/or for specific medical substance(s). External device 110 may transmit, via communications circuitry 304, data corresponding to ML models to hearing instruments 102. In some examples, external device 100 may transmit data corresponding to ML models corresponding to specific medical substances included in the medicinal history received by external device 110.

Processor(s) 302 may receive types and dosages of medical substances ingested by the user. For example, processor(s) 302 may receive, via communications circuitry 304 or via UI 306, medicinal history of the user. The medicinal history may include the types and dosages of medical substances ingested by the user within a period of time. The period of time may vary between medical substances and may be based on a maximum duration of substance addition and/or substance withdrawal after ingestion of the medical substance. Processor(s) 302 may retrieve, from the medicinal history, the types of dosages of the medical substances. Processor(s) 302 may select, from the medical substances included in the medicinal history of the user, one or more medical substances that may cause the user to experience symptoms of medical substance addiction, symptoms of medical substance withdrawal, or a medical overdose. Processor(s) 302 may select one or more medical substances from outside of the medical substances included in the medicinal history. For example, processor(s) 302 may select opioids as a medical substance regardless of whether the user has previously ingested opioids.

Processor(s) 302 may transmit, via communications circuitry 304, data corresponding to threshold conditions and ML models for each of the selected medical substances to hearing instruments 102. Processor(s) 302 may receive, via communications circuitry 304, a notification from hearing instruments 102 that the user has a threshold likelihood of experiencing one or more scenarios in response to ingestion of one or more medical substances. Processor(s) 302 may verify that the user is experiencing or will experience the one or more scenarios. Processor(s) 302 may receive, from sensor(s) coupled to or in communication with external device 110, sensor data corresponding to physiology of the user (e.g., visual signals, audio signals, physiological signals). For each scenario included in the notification from hearing instruments 102, processor(s) 302 may verify the notification from hearing instrument 102 based on a determination that the sensor data corresponds to symptoms or behavior of the user that are consistent with the scenario (e.g., symptoms/behaviors consistent with a medical overdose, symptoms/behaviors consistent with medical substance withdrawal).

Processor(s) 302 may, in response to receiving the notification from hearing instruments 102, output an alert to the user via UI 306. Processor(s) 302 may cause UI 306 to output a visual, auditory, and/or tactile signal to the user to alert the user that the user has a threshold likelihood of experiencing one or more scenarios. For example, processor(s) 302 may cause UI 306 to output an alert indicating that the user has a threshold likelihood of experiencing a medical overdose. The alert may indicate the medical substance causing or is likely to cause the scenario. The alert may indicate a likelihood that the user is experiencing a scenario (e.g., an 80% likelihood that the user is experiencing a medical overdose). Processor(s) 302 may output, as a part of the alert, recommended actions for the user. The recommended actions may include, but are not limited to, a recommendation to contact a medical care provider, a recommendation to seek emergency care, or a recommendation to ingest a medical substance to mitigate the effects of the scenario (e.g., naloxone).

Processor(s) 302 may cause communications circuitry 304 output a notification to a computing device, system, or cloud computing environment of one or more third parties in response to receiving the notification from hearing instruments 102. Communications circuitry 304 may transmit the notification to the computing device, system, or cloud computing environment via network 112. Third parties may include, but are not limited to, family member(s) of the user, a caretaker of the user, medical care providers, EMS providers, or the like. The notification may indicate, for each scenario the user may be experiencing as indicated in the notification from hearing instruments 102, a likelihood that the user may experience the scenario, a medical substance causing or is likely to cause the scenario, a time when hearing instruments 102 determined the likelihood, or the like. The notification may include a location of the user, e.g., to facilitate the rendering of medical aid to the user.

UI 306 may include one or more components configured to receive instructions from and/or present information to the user. UI 306 may include, but are not limited to, display screens, camera, microphones, haptic feedback components, speakers, or the like. In some examples, UI 306 may receive the medicinal history of the user and may transmit the user input to processor(s) 302, e.g., for determination of the data to be transmitted to hearing instruments 102. UI 306 may output a notification (e.g., a visual, auditory, and/or tactile signal) to the user indicating the likelihoods that the user is experiencing or will experience one or more scenarios in response to a medical substance as well as recommended actions for the user.

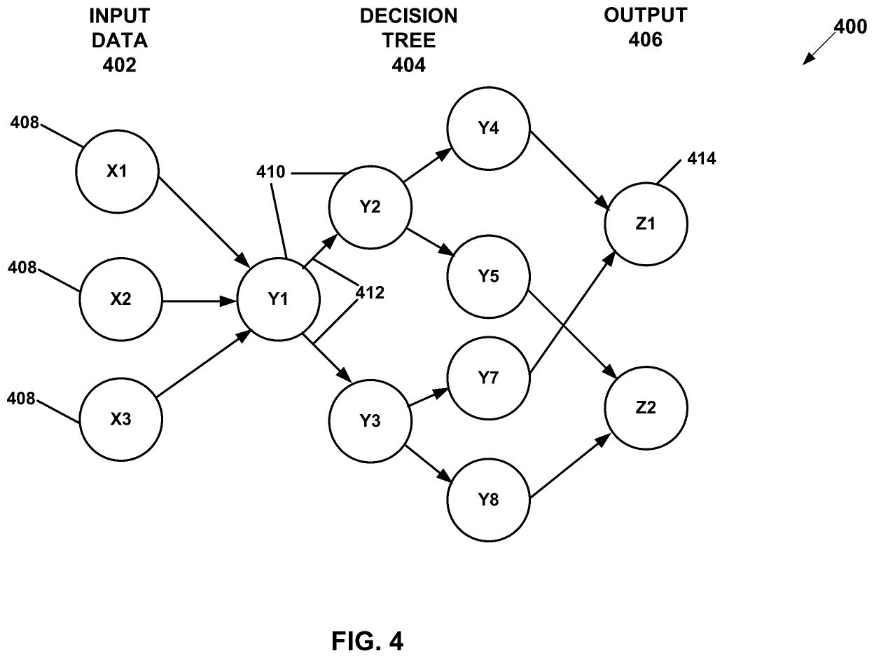

FIG. 4 is a conceptual diagram illustrating an example machine learning (ML) model 400 stored in the example hearing instrument system 100 of FIG. 1. In some examples, as illustrated in FIG. 4, ML model 400 may be a decision tree model. In other examples, ML model 400 may be an artificial neural network (ANN) model, a distributed neural network (DNN) model, a support vector machine (SVM) model, or other type of ML model.

ML model 400 may include a decision tree 404 configured to receive input data 402 and to determine outputs 406. Different ML models may include the same or different decision trees. Each decision tree may be trained to determine a likelihood that the user is experiencing a specific scenario for a specific medical substance based on input data 402. For example, system 100 may apply a first ML model with a first decision tree to determine a likelihood of a medical overdose in response to a first medical substance and apply a second ML model with a second decision tree to determine a likelihood of a medical overdose in response to a second medical substance. In some examples, system 100 may apply a third ML model with a third decision tree to determine a likelihood of symptoms of addition to the first medical substance. In some examples, each ML model 400 may be configured to determine likelihoods for two or more scenarios and/or in response to two or more medical substances.

Input data 402 may include one or more input values 408 (e.g., X1-X3). Each input value 408 may correspond to a different physiological parameter. For example, as illustrated in FIG. 4, input value X1 may correspond to a respiration pattern of the user, input value X2 may correspond to a speech pattern of the user, and input value X3 may correspond to occurrences of choking or vomiting experienced by the user. While in some examples, as illustrated in FIG. 4, input data 402 includes 3 input values 408, input data 402 may include one, two, or four or more inputs values 408 for other ML models 400.

As a part of applying ML model 400, system 100 may enter input values 408 (e.g., input values X1-X3) of input data 402 into decision tree 404 for ML model 400. Decision tree 404 may be defined by a plurality of nodes 410, e.g., nodes Y1-Y8 as illustrated in FIG. 4. Each node 410 may be connected to one or more other nodes 410 via edges 412. Each node 410 may represent a condition and edges 412 may represent possible pathways to subsequent nodes 410 based on the satisfaction, or lack thereof, of the condition. For example, node Y1 is connected to nodes Y2, Y3 via edges 412. Based on a determination that input values 408 satisfy node Y1, decision tree 404 may instruct system 100 to progress along edge 412 to node Y2. Based on a determination that input values 408 do not satisfy node Y1, decision tree 404 may instruct system 100 to progress along edge 412 to node Y3.

For each node 410, system 100 may determine whether input data 402 satisfies the condition for the node 410. Each condition may correspond to one, all, or any combination of input values 408. Each condition may result in two or more results, e.g., such that each respective node 410 is connected to two or more subsequent nodes 410 via edges 412. Each subsequent node 410 may correspond to a subsequent condition and may be further linked to additional nodes 410 via edges 412. The conditions for nodes 410, the relationships between nodes 410, and the number of nodes 410 may be determined as a part of training ML model 400.

System 100 may progress along nodes 410 and edges 412 of decision tree 404 until system 100 arrives at one of output values 414 (e.g., outputs Z1, Z2) of outputs 406. Each output value 414 may correspond to a different likelihood value (e.g., a specific percentage, a specific ratio, a specific likelihood score). For example, output Z1 may correspond to a likelihood value of 60% and output Z2 may correspond to a likelihood value of 50%. Outputs 406 may include two or more output values 414, each output value 414 corresponding to a different likelihood value. System 100 may arrive at each output value 414 via a single path along nodes 410 and edges 412 of decision tree 404 or via multiple paths. For example, as illustrated in FIG. 4, system 100 arrive at output Z1 via a first path from Y1 to Y2 and from Y2to Y4 or via a second path from Y1 to Y3 and from Y3 to Y7. While FIG. 4 illustrates ML model 400 being configured to navigate system 100 from input data 402 to any output value 414 of outputs 406 via three linked nodes 410, other ML models 400 may link input data 402 to each output value 414 via four or more nodes 410 (e.g., tens of nodes 410, hundreds of nodes 410).

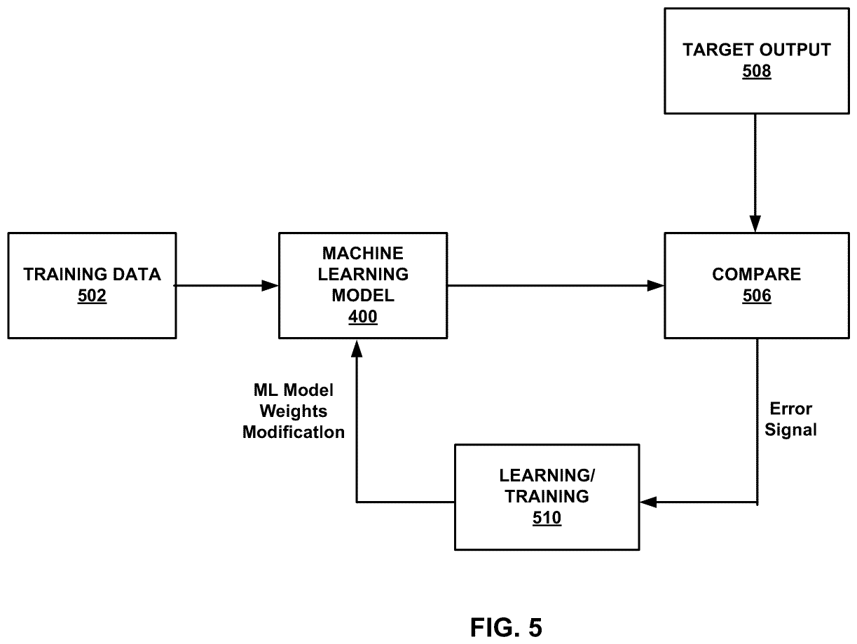

FIG. 5 is a block diagram illustrating an example process of training the example ML model 400 of FIG. 4. ML model 100 may be implemented using any number of models for supervised and/or reinforced learning, such as but is not limited to, an ANN model, a DNN model, naïve Bayes network, a SVM model, or a k-nearest neighbor model. In some examples, processing circuitry (e.g., processor(s) 302) of external device 110 or other computing devices, systems, or cloud computing environments connected to network 112 initially trains ML model 400 based on training data 502. Training data 502 may be sourced from the user and/or one or more other individuals. Training data 502 may include example physiological parameter values for each individual and corresponding labels on whether the individual experienced a specific scenario (e.g., symptoms of medical substance addiction, symptoms of medical substance withdrawal, medical overdose, imminent medical overdose), e.g., as labeled by an expert. The expert may also label the medical substance(s) that caused the individual to experience each scenario.

The processing circuity may generate ML model 400 (e.g., decision tree 404 of ML model 400). The processing circuitry may generate decision tree 404 by randomly generating nodes 410 and edges 412 between nodes 410. The processing circuitry may randomly generate conditions based on the physiological parameters within training data 502 and randomly assign the conditions to nodes 412. In some examples, physiological parameters in training data 502 may include physiological parameters that may not be used as inputs by the completed decision tree 404.

A prediction or classification by ML model 400 in response to example input data 402 may be compared 506 to target output 508, e.g., as determined based on the label. Based on an error signal representing the comparison, the processing circuitry implementing a learning/training function 510 may send a modification to weights of ML model 400 and/or to nodes 410 and edges 412 of decision tree 404. The error signal may indicate an amount of deviation between the prediction or classification and target output 508. The modifications may include, but are not limited to, the inputted physiological parameters for input data 402 (e.g., from training data 502), the number of nodes 410, the connections between nodes 410 via edges 412, the conditions for nodes 410, a number of nodes 410 linking input data 402 to outputs 406, a weight assigned to each inputted physiological parameter, a weight assigned to each node 410, pathways from specific nodes 410 to specific outputs 406 (e.g., from a root node Y1 to output Z1), or the like.

The processing circuitry may then input training data 502 to the modified ML model 400 and compare 506 the outputted prediction or classification to target output 508 to generate a new error signal. The processing circuitry may iteratively compare 506 predictions or classifications of ML model 400 to target output 508, determine an error signal between the predictions or classifications and target output 508, and modify, based on the error signal, ML model 400 via learning/training function 510 until the processing circuitry determines that the prediction or classifications of ML model 400 (e.g., of decision tree 404) satisfies a threshold condition. The threshold condition may include, but is not limited to, a determination that a threshold number of predictions or classification deviate from target output 508 by less than or equal to a threshold percentage (e.g., 10%, 5%, 1%, 0.01%).

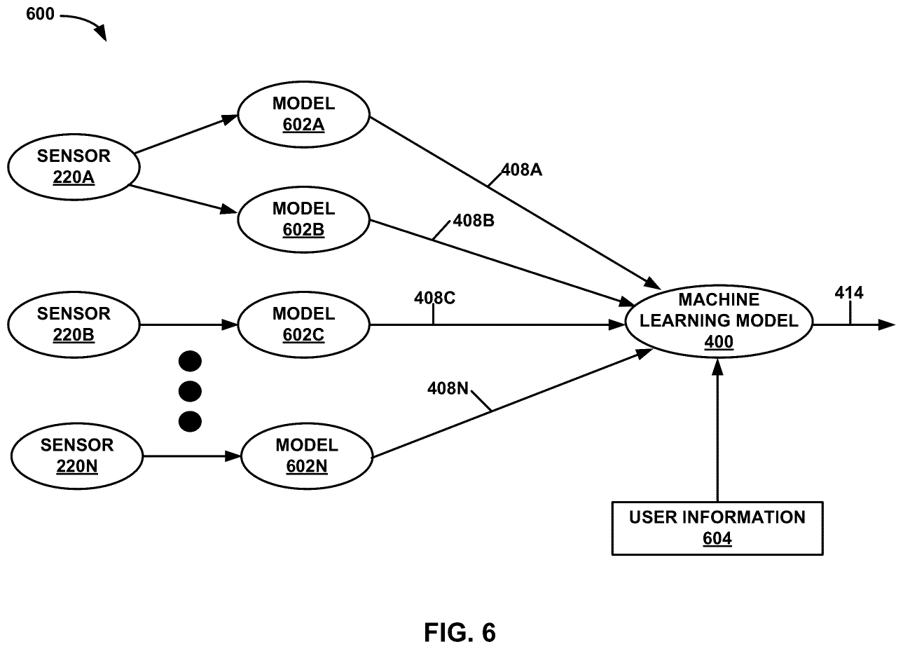

FIG. 6 is a block diagram illustrating an example process 600 of applying ML model 400 of FIG. 4. In some examples, system 100 may directly input physiological signals sensed by sensor(s) 220 or received by system 100 into ML model 400 to generate a prediction or classification by ML model 400. In some examples, as illustrated in FIG. 6, system 100 applies one or more models 602A-N (collectively referred to herein as “models 602”) to physiological signals sensed by sensors 220A-220N (collectively referred to herein as sensor(s) 220) to generate input values 408A-408N (collectively referred to herein as “inputs values 408”) for ML model 400.

Sensor(s) 220 may include accelerometers, gyroscopes, IMUs, heartrate monitors, PPG sensors, temperature sensors, microphones, oximeters, and the like. In the example process 600 illustrated in FIG. 6, system 100 may input sensed physiological signals from one or more of sensor(s) 220 into one or more models 602 to generate inputs values 408 for ML model 400. System 100 may input physiological signals from different sensor(s) 220 may into different models 602 or to the same models 602. For example, as illustrated in FIG. 6, system 100 inputs sensed physiological signals from sensor 220A to model 602A and model 602B In some examples, one or more of sensor(s) 220 may sense more than one type of physiological signals. In such examples, system 100 may transmit the same or different types of physiological signals from the one or more of sensor(s) 220 to each of two or more different models 602.

Each of models 602 may be trained and/or configured to output a determination based at least in part on the inputted sensed physiological signals. System 100 may apply one or more models 602 to the inputted sensed physiological signals to generate corresponding outputs from the one or more models 602. For example, each of models 602 may be configured to determine, based at least in part on the inputted sensed physiological signals, whether the user is experiencing a specific symptom, a severity of a symptom experienced by the user, and/or whether the sensed physiological signals deviate from an expected physiological signal value (e.g., based on prior sensed physiological signal values, based on an average or median physiological signal value) by at least a threshold amount. In some examples, one or more of models 602 may be configured to determine one or more physiological parameters of the user based on the inputted sensed physiological signals. Each of models 602 may output the determination based on sensed physiological signals from one or two or more sensor(s) 220. Models 602 may make determinations regarding different symptoms, different input physiological signals, different physiological parameters, or the like. Each of models 602 may be a rule-based model or a machine learning model (e.g., a neural network model). Each of models 602 may be trained in a same or similar manner as the training of ML model 400 as previously described with respect to FIG. 5.

System 100 may input the outputs from models 602 as input values 408 for ML model 400. Input values 408 may include, but are not limited to, physiological parameter values or determinations of occurrence and/or severities of physiological symptoms. In some examples, system may input user information 604 into ML model 400, e.g., as one of input values 408. User information 604 may include, but are not limited to, medicinal history of the user and/or information on substances recently ingested by or suspected to have been ingested by the user. System 100 may apply ML model 400 to input values 408 and/or user information 604 to output a determination 414. Determination 414 may indicate a likelihood that the user is experiencing a medical scenario, the type of the medical scenario, the severity of the medical scenario, and/or recommended courses of action to treat the medical scenario.

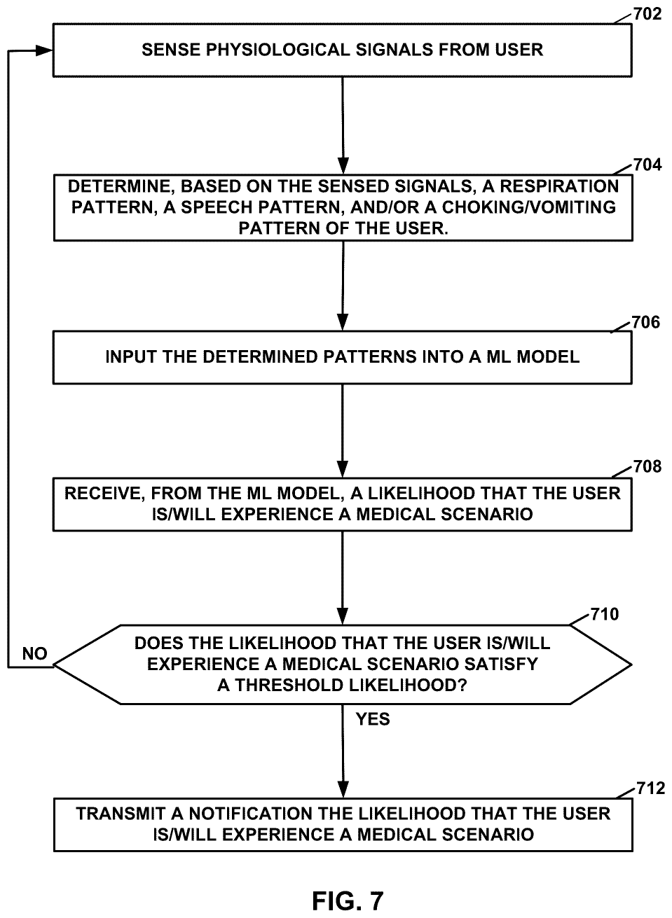

FIG. 7 is a flow diagram illustrating an example process of determining a likelihood that a user is experiencing a medical scenario. While FIG. 7 is primarily described in reference to determining the likelihood that the user is experiencing a medical overdose, the example process described herein may be applied by hearing instrument system 100 to determine whether the user is experiencing other medical scenarios (e.g., symptoms of medical substance addiction, symptoms of medical substance withdrawal). Additionally, while the example process illustrated in FIG. 7 is primarily described below as being performed by hearing instruments 102 of hearing instrument system 100, the example process may be performed by other computing devices, systems, or cloud computing environments of system 100 or connected to system 100 via network 112.

Hearing instruments 102 may sense physiological signals from a user (702). Hearing instruments 102 may include sensor(s) 220 including, but are not limited to, accelerometers, gyroscopes, IMUs, microphones, oximeters, or PPG sensors. When the user wears hearing instruments 102 in, on, or about one or more ears of the user, sensor(s) 220 may sense physiological signals from the user. Physiological signals may include, but are not limited to, motion signals, PPG signals, audio signals, oximetry signals, or the like. Sensor(s) 220 may sense physiological signals constantly, periodically (e.g., once every ten seconds, twenty seconds, one minute), or in response to an instruction from external device 110.

Hearing instruments 102 may determine, based on the sensed physiological signals, a respiration pattern, a speech pattern, and a choking/vomiting pattern of the user (704). The respiration pattern may include but are not limited to, a delay between exhalation and inhalation by the user or an intra-breath spacing between temporally adjacent breaths by the user. The speech pattern may include, but are not limited to, a clarity of speech by the patient or a rate of speech by the patient. The choking/vomiting pattern of the user may include a frequency and/or a duration of choking or vomiting by the user. Hearing instruments 102 may determine respiration of the user and thereby the respiration pattern of the user based on sensed motion signals and/or PPG signals. Hearing instruments 102 may determine speech pattern of the user and/or instances of choking or vomiting by the user via sensed motion signals (e.g., to determine movement of the head of the user) or via sensed audio signals (e.g., to detect speech, choking, or vomiting by the user).

Hearing instruments 102 may input the determined patterns into a ML model (e.g., ML model 400) (706). Hearing instruments 102 may select ML models from a plurality of ML models stored in system 100 based on types of medical substances hearing instrument 102 is configured to monitor. Hearing instruments 102 may select ML models based on medical substances currently or previously ingested by the user. Hearing instruments 102 may determine medical substances currently or previously ingested by the user based on the medicinal history of the user stored or received by system 100. Medical substances may include, but are not limited to, pharmaceutical substances, illicit substances, therapeutic substances, or the like. Hearing instruments 102 may select ML models based on one or more types of scenario hearing instruments 102 are configured to monitor (e.g., medical overdose, imminent medical overdose, symptoms of medical substance overdose, symptoms of medical substance withdrawal).

Hearing instruments 102 may receive, from the ML model, a likelihood that the user is/will experience a medical scenario (708). Hearing instruments 102 may apply the ML model to output the likelihood of a medical scenario (e.g., of a medical overdose). The ML model may be a decision tree model, an ANN model, a DNN model, an SVM model, or other ML models. The ML model may be trained via a training set including values for respiration, speech, and/or choking/vomiting patterns and corresponding occurrences or non-occurrences of medical overdose. The likelihood of the medical scenario may be represented in terms of a percentage, a ratio, or a likelihood score. Hearing instruments 102 may apply different ML models for different medical substances and/or to determine the likelihood of occurrences of different medical scenarios.

Hearing instruments 102 may determine whether the likelihood that the user is/will experience a medical scenario satisfies a threshold likelihood (710). The threshold likelihood may represent a minimum likelihood that the user may require medical care for any given scenario and/or medical substance. Hearing instruments 102 may compare, for each combination of scenario or medical substance, the determined likelihood against a different threshold likelihood corresponding to the specific scenario and/or medical substance. Hearing instruments 102 may determine that the determined likelihood satisfies the corresponding threshold likelihood based on a determination that the likelihood is greater than or equals to the threshold likelihood (e.g., the determined ratio is greater than or equal to the threshold ratio, the determined percentage is greater than or equal to the threshold percentage, the determined likelihood score is greater than or equal to the threshold likelihood score).

Based on a determination that the determined likelihood does not satisfy the threshold likelihood (“NO” branch of 710), hearing instruments 102 may continue to sense physiological signals from the user (702). Based on a determination that the determined likelihood satisfies the threshold likelihood (“YES” branch of 710), hearing instruments 102 may transmit a notification the likelihood that the user is/will experience a medical scenario (712). Hearing instruments 102 may transmit the notification to external device 110, e.g., to alert the user to the likelihood of the medical scenario. The notification may include one or more likelihood values, the type of the medical scenario corresponding to each likelihood value, and/or the medical substance(s) causing and/or contributing to each medical scenario. Hearing instruments 102 may transmit the notification, via external device 110, to one or more computing devices, systems, or cloud computing environments in communication with network 112, e.g., to alert caretakers, medical care providers, or EMS providers and request medical aid for the user.

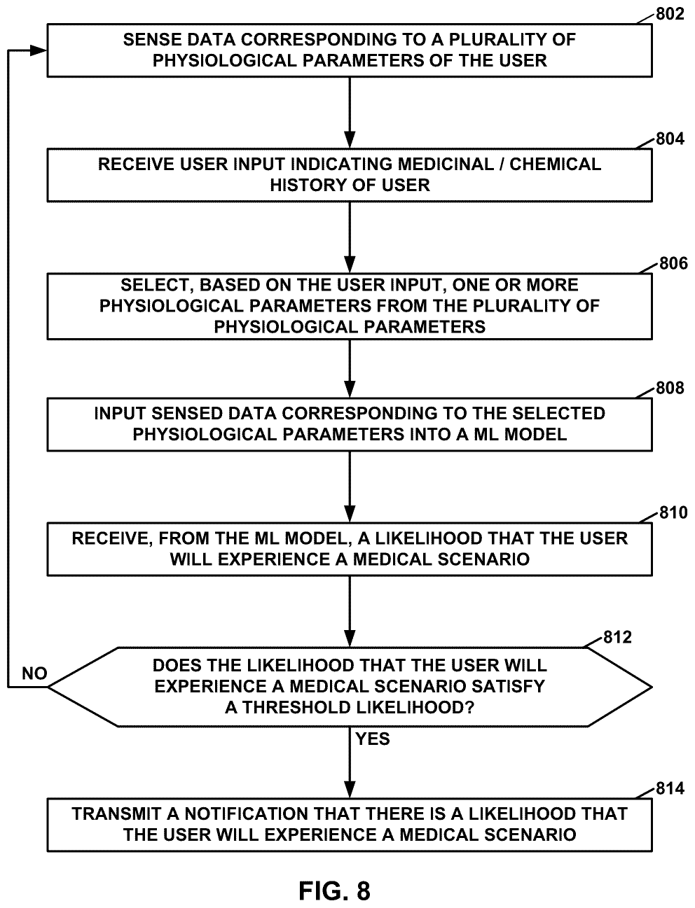

FIG. 8 is a flow diagram illustrating another example process of determining a likelihood that a user is experiencing a medical scenario. Medical scenarios may include, but are not limited to, symptoms of a medical substance addiction, symptoms of a medical substance withdrawal, a medical overdose, or an imminent medical overdose. While the example process illustrated in FIG. 8 is described below primarily with reference to hearing instruments 102 of hearing instrument system 100 of FIG. 1, the example process may be performed by other computing devices, systems, or cloud computing environments that are a part of or are connected to system 100 (e.g., external device 110, computing devices and/or systems connected to network 112).

Hearing instruments 102 may sense data corresponding to a plurality of physiological parameters of the user (802). Physiological parameters may include, but are not limited to, a respiration rate, a respiration pattern, a heart rate, a blood oxygen saturation, speech pattern, instances and/or frequency of choking, instances and/or frequency of vomiting, instances and/or frequency of swallowing, balance, gait, or other physiological behaviors that correspond to effects of medical substances. When the user wears hearing instruments 102 in, on, or about one or more ears, sensor(s) 220 within hearing instruments 102 may sense physiological signals and determine values for the plurality of physiological parameters based on the sensed physiological signal data. Sensor(s) 220 may include, but are not limited to, an accelerometer, a gyroscope, an IMU, a PPG sensor, a microphone, or an oximeter.

Hearing instruments 102 may receive user input indicating medicinal history of the user (804). The medicinal history of the user may indicate the types and dosages, for any medical substances currently or previously ingested by the user. The medicinal history may also indicate a frequency of use and a last use of each medical substance. Medical substances may include, but are not limited to, pharmaceutical substances (prescription medication, over-the-counter medication), therapeutic substances, illicit substances, alcohol, or other chemical substances which may have one or more chemical or pharmaceutical effects on the user. The user or a third party (e.g., a family member, a caretaker, a clinician) may input the medicinal history of the user into system 100 via UI 306 or communications circuitry 304 on external device 110. In some examples, external device 110 transmits at least a portion of the received user input to hearing instruments 102 (e.g., the types of medical substances ingested by the user) to cause hearing instruments 102 to monitor possible medical scenarios resulting from specific medical substances. In some examples, external device 110 selects, based on the medical substances ingested by the user, possible medical scenarios for hearing instruments 102 to monitor and transmits instructions to hearing instruments 102 to monitor the selected medical scenarios.

Hearing instruments 102 may select, based on the user input, one or more physiological parameters from the plurality of physiological parameters (806). The ingestion of different medical substances may have different effects on different combinations of physiological parameters. As such, each medical substance may correspond to a different combination of physiological parameters. Based on the user input, hearing instruments 102 may select or receive selected medical scenarios to monitor. Each selected medical scenario may correspond to a different medical substance or combination of medical substances than another selected medical scenario. The selected medical scenarios may not correspond to effects on all of the plurality of physiological parameters and each selected medical scenario may correspond to a same or different combination of physiological parameters than another selected medical scenario. Hearing instruments 102 may select the one or more physiological parameters corresponding to the selected medical scenarios from the plurality of physiological parameters.

Hearing instruments 102 may input sensed data corresponding to the selected physiological parameters into a ML model (e.g., ML model 400) (808). The sensed data may be raw physiological signal data or may be converted from the raw physiological signal data into corresponding physiological parameter values. The ML model may correspond to a selected medical scenario and may be configured to determine a likelihood that the user is experiencing the medical scenario based on the inputted sensed data. Each ML model may correspond to a different medical scenario and/or correspond to different medical substance(s). Hearing instruments 102 may store a plurality of ML models and may select and apply one or more ML models based on the selected medical scenarios. In some examples, hearing instruments 102 receive ML models from external device 110 based on the selected medical scenarios and apply the received ML models.

Hearing instruments 102 may receive, from the ML model, a likelihood that the user will experience a medical scenario (810). The likelihood may be represented in terms of a percentage, a ratio, or a likelihood score. Hearing instruments 102 may determine whether the likelihood that the user will experience a medical scenario satisfies a threshold likelihood (812). The threshold likelihood may represent a minimum likelihood at which the user may seek medical aid. The threshold likelihood may be determined by a clinician and may be adjusted (e.g., by system 100, by the clinician) in response to changes in the physiological parameters of the user over time. Hearing instrument 102 may determine that the likelihood satisfies the threshold likelihood based on a determination that the determined likelihood is greater than or equal to the threshold likelihood.

In response to a determination that the likelihood does not satisfy the threshold likelihood (“NO” branch of 812), hearing instruments 102 may continue to sense data corresponding to a plurality of physiological parameters of the user (802). In response to a determination that the likelihood satisfies the threshold likelihood (“YES” branch of 812), hearing instruments 102 may transmit a notification that there is a likelihood that the user is experiencing a medical scenario (814). Hearing instruments 102 may transmit the notification to computing devices, systems, or cloud computing environments connected to network 112 via external device 110. The notification may indicate a type of the medical scenario, possible medical substances associated with the medical scenario, the determined likelihood, the severity of the medical scenario, and/or a request for medical aid.

Hearing instruments 102 may transmit the notification to different individuals based on a severity of the medical scenario. For example, hearing instruments 102 transmits the notification to a caretaker or a family member for a less severe medical scenario and to a medical care provider or EMS provider in response to a more severe medical scenario. The severity of the medical scenario may be based on the value of the determined likelihood, e.g., whether the determined likelihood satisfies one or more threshold severity likelihoods. In some examples, the severity of the medical scenario may be based on the ML model. For example, hearing instruments 102 may monitor a medical scenario at multiple levels of severity via a plurality of ML models, each ML model configured to determine a likelihood that the user is experiencing the medical scenario at a different level of severity.

It is to be recognized that depending on the example, certain acts or event of any of the techniques described herein can be performed in a different sequence, may be added, merged, or left out altogether (e.g., not all described acts or events are necessary for the practice of the techniques). Moreover, in certain examples, acts or events may be performed simultaneously, e.g., through multi-threaded processing, interrupt processing, or multiple processors, rather than sequentially.

In some examples, system 100 comprises: an ear-wearable device 102A configured to be worn in, on, or about an car of a user, the car-wearable device 102A comprising: a sensor 220 configured to sense one or more physiological parameters of the user; and memory (e.g., storage device(s) 200, storage device(s) 300); and a processing system (e.g., processor(s) 206, 215, 302) configured to: retrieve, from car-wearable device 102A, the one or more sensed physiological parameters; retrieve, from the memory, a machine learning (ML) model 400, wherein the ML model 400 is configured to be trained via a training set comprising a plurality of physiological parameter values and a corresponding plurality of symptoms of a medical overdose; apply the ML model 400 to the one or more sensed physiological parameters to determine whether a likelihood that the user is experiencing or will experience the medical overdose; and based on a determination that the user is experiencing symptoms of the medical overdose, transmit a notification to a computing device (e.g., external device 110) indicating that the likelihood that the user is experiencing or will experience the medical overdose.

In some examples, system 100 is configured to perform a method comprising: sensing, via a sensor 220 disposed within an car-wearable device 102A configured to be worn in, on, or about an car of a user, one or more physiological parameters of the user; applying, by processing circuitry (e.g., processor(s) 206, 215) of the car-wearable device 102A, a machine learning (ML) model 400 to the one or more physiological parameters to determine a likelihood that the user will experience a medical overdose; and transmitting, by the processing circuitry and via communications circuitry (e.g., wireless communication system 202) of the car-wearable device 102A, a notification indicating the likelihood that the user will experience the medical overdose.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer readable storage medium which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection is properly termed a computer readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some respects, the functionality described herein may be provided within dedicated hardware and/or software modules. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

The following is a non-limiting list of examples that are in accordance with one or more aspects of this disclosure.

Example 1: an ear-wearable device comprising: a sensor configured to sense one or more physiological parameters of a user of the car-wearable device; communications circuitry; and processing circuitry configured to: apply a machine learning (ML) model to the one or more sensed physiological parameters to determine whether the user is experiencing symptoms of a medical scenario, wherein the ML model is configured to be trained via a training set comprising a plurality of physiological parameter values and a corresponding plurality of symptoms of the medical scenario; and based on a determination that the user is experiencing symptoms of the medical scenario, cause the communications circuitry to transmit a notification indicating that the user is experiencing the symptoms of the medical scenario.

Example 2: the car-wearable device of example 1, wherein the ML model comprises a decision tree model.

Example 3: the car-wearable device of any of examples 1 and 2, wherein the medical scenario comprises at least one of: a medical overdose; a medical substance withdrawal; or a medical substance addiction;

Example 4: the car-wearable device of any of examples 1-3, wherein the one or more physiological parameters comprises at least one of: a respiration pattern of the user; a speech pattern of the user; instances of vomiting by the user; or instances of choking by the user.

Example 5: the car-wearable device of example 4, wherein the respiration pattern comprises one or more of: a delay between an exhalation and an inhalation by the user; or an intra-breath spacing between temporally adjacent breaths by the user.

Example 6: the car-wearable device of any of examples 1-5, wherein the one or more physiological parameters comprises at least one of: a change in a respiration rate of the user; a change in a respiration pattern of the user; or a change in a speech pattern of the user.

Example 7: the car-wearable device of any of claims 1-6, wherein the processing circuitry is further configured to: receive, via the communications circuitry, user input indicating a medicinal history of the user; and select, based on the received user input, the one or more physiological parameters from a plurality of available physiological parameters, wherein the one or more physiological parameters are indicative of symptoms of the medical scenario from a medical substance listed in the medicinal history of the user.

Example 8: the car-wearable device of example 7, wherein the medicinal history comprises: a type of a medical substance used by the user within a period of time; and a dosage of the medical substance used by the user.

Example 9: the car-wearable device of any of examples 7 and 8, further comprising a memory configured to store a plurality of ML models, and wherein the processing circuitry is configured to: select, based on the medical substance listed in the medicinal history of the user, the ML model from the plurality of ML models, wherein each ML model of the plurality of ML models is configured to determine a likelihood that the user is experiencing the medical scenario from a corresponding medical substance.

Example 10: the car-wearable device of any of examples 1-9, wherein the sensor comprises one or more of an accelerometer, an inertial measurement unit (IMU), a microphone, a photoplethysmography (PPG) sensor, or an oximeter.

Example 11: the car-wearable device of any of examples 1-10, wherein to cause the communications circuitry to transmit the notification indicating that the user is experiencing the symptoms of the medical scenario, the processing circuitry is configured to: receive, via the communications circuitry, sensor data confirming that the user is experiencing the symptoms of the medical scenario; and cause the communications circuitry to transmit the notification based on receipt of the sensor data.

Example 12: the car-wearable device of example 11, wherein the sensor data comprises one or more of: a body temperature of the user; a change in the body temperature of the user; a posture of the user; a change in the posture of the user; a heart rate of the user; or a change in the heart rate of the user.

Example 13: a system comprising: an car-wearable device configured to be worn in, on, or about an car of a user, the car-wearable device comprising: a sensor configured to sense one or more physiological parameters of the user; and memory; and a processing system configured to: retrieve, from car-wearable device, the one or more sensed physiological parameters; retrieve, from the memory, a machine learning (ML) model, wherein the ML model is configured to be trained via a training set comprising a plurality of physiological parameter values and a corresponding plurality of symptoms of a medical scenario; apply the ML mode to the one or more sensed physiological parameters to determine whether a likelihood that the user is experiencing or will experience the medical scenario; and based on a determination that the user is experiencing symptoms of the medical scenario, transmit a notification to a computing device indicating that the likelihood that the user is experiencing or will experience the medical scenario.

Example 14: the system of example 13, wherein the ML model comprises a decision tree model.

Example 15: the system of any of examples 13 and 14, wherein the medical scenario comprises at least one of: a medical overdose; a medical substance withdrawal; or a medical substance addiction.

Example 16: the system of any of examples 13-15, wherein the processing system is configured to: receive, from the ML model, a value corresponding to the likelihood that the user will experience the medical scenario; compare the received value against a threshold value stored in the memory, wherein the threshold value corresponds to a threshold likelihood that the user will experience the medical scenario; and based on a determination that the received value satisfies the threshold value, transmit the notification to the computing device.

Example 17: the system of any of examples 13-16, wherein the one or more physiological parameters comprises one or more of: a respiration pattern of the user; a speech pattern or the user; instances of vomiting by the user; or instances of choking by the user.

Example 18: the system of example 17, wherein the respiration pattern comprises one or more of: a delay between an exhalation and an inhalation by the user; or an intra-breath spacing between temporally adjacent breaths by the user.

Example 19: the system of any of examples 13-18, wherein the one or more physiological parameters comprises at least one of: a change in a respiration rate of the user; a change in a respiration pattern of the user; or a change in a speech pattern of the user.

Example 20: the system of any of examples 13-19, wherein the processing system is configured to: receive a medicinal history of the user; and select, based on the medicinal history, the one or more physiological parameters from the plurality of physiological parameters, wherein the one or more physiological parameters are indicative of an occurrence of a possible medical scenario from a medical substance listed in the medicinal history.

Example 21: the system of example 20, wherein the medicinal history comprises: a type of each medical substance used by the user within a period of time; and a dosage of each medical substance used by the user.

Example 22: the system of any of examples 20 and 21, wherein the memory is configured to store a plurality of ML models, wherein each ML model is configured to determine a likelihood that the user is experiencing a medical scenario of a corresponding medical substance, and wherein the processing system is configured to select the ML model from the plurality of ML model based on the medical substance listed in the medicinal history.

Example 23: the system of any of examples 13-22, wherein the sensor comprises one or more of an accelerometer, an inertial measurement unit (IMU), a microphone, a photoplethysmography (PPG) sensor, or an oximeter.

Example 24: a method comprising: sensing, via a sensor disposed within an car-wearable device configured to be worn in, on, or about an car of a user, one or more physiological parameters of the user; applying, by processing circuitry of the car-wearable device, a machine learning (ML) model to the one or more physiological parameters to determine a likelihood that the user will experience a medical scenario; and transmitting, by the processing circuitry and via communications circuitry of the car-wearable device, a notification indicating the likelihood that the user will experience the medical scenario.

Example 25: the method of example 24, wherein the ML model comprises a decision tree model.

Example 26: the car-wearable device of any of examples 24 and 25, wherein the medical scenario comprises at least one of: a medical overdose; a medical substance withdrawal; or a medical substance addiction.

Example 27: the method of any of examples 24-26, further comprising: receiving, by the processing circuitry and from the ML model, a value corresponding to the likelihood that the user will experience the medical scenario; comparing, by the processing circuitry, the received value against a threshold value stored in memory of the car-wearable device, wherein the threshold value corresponds to a threshold likelihood that the user will experience the medical scenario; and based on a determination that the received value satisfies the threshold value, causing the communications circuitry to transmit the notification.

Example 28: the method of any of examples 24-27, wherein the one or more physiological parameters comprises at least one of: a respiration pattern of the user; a speech pattern of the user; instances of vomiting by the user; or instances of choking by the user.

Example 29: the method of example 28, wherein the respiration pattern comprises one or more of: a delay between an exhalation and an inhalation by the user; or an intra-breath spacing between temporally adjacent breaths by the suer.

Example 30: the method of any of examples 24-29, wherein the one or more physiological parameters comprises at least one of: a change in a respiration rate of the user; a change in a respiration pattern of the user; or a change in a speech pattern of the user.

Example 31: the method of any of examples 24-30, further comprising: receiving, by the processing circuitry, user input indicating a medicinal history of the user; and selecting, based on the medicinal history, the one or more physiological parameters from a plurality of physiological parameters, wherein the one or more physiological parameters are indicative of symptoms of the medical scenario from a medical substance listed in the medicinal history of the user.

Example 32: the method of examples 31, wherein the medicinal history comprises: a type of each medical substance used by the user with a period of time; and a dosage of each medical substance used by the user.

Example 33: the method of any of examples 31 and 32, further comprising: selecting, by the processing circuitry and from a plurality of ML models stored in memory of the car-wearable device, the ML model, wherein each ML model of the plurality of ML models is configured to determine a likelihood that the user is experiencing a medical scenario from a corresponding medical substance.

Example 34: the method of any of examples 24-33, wherein the sensor comprises one or more of an accelerometer, an inertial measurement unit (IMU), a microphone, a photoplethysmography (PPG) sensor, or an oximeter.

Example 35: the method of any of examples 24-34, further comprising: receiving by the processing circuitry and via the communications circuitry, sensor data indicating that the user is experiencing a symptom of the medical scenario; and transmitting, by the processing circuitry and via the communications circuitry, the notification based on receipt of the sensor data.

Example 36: the method of example 35, wherein the sensor data comprises one or more of: a body temperature of the user; a change in the body temperature of the user; a posture of the user; a change in the posture of the user; a heart rate of the user; or a change in the heart rate of the user.

Example 37: a computer-readable medium comprising instructions that, when executed, causes processing circuitry of an ear-wearable device to perform the method of any of examples 24-34.

Various examples have been described. These and other examples are within the scope of the following claims.

What is claimed is:

1. An ear-wearable device comprising:
a sensor configured to sense one or more physiological parameters of a user of the ear-wearable device;
communications circuitry; and
processing circuitry configured to:
apply a machine learning (ML) model to the one or more sensed physiological parameters to determine whether the user is experiencing symptoms of a medical scenario, wherein the ML model is configured to be trained via a training set comprising a plurality of physiological parameter values and a corresponding plurality of symptoms of the medical scenario; and
based on a determination that the user is experiencing symptoms of the medical scenario, cause the communications circuitry to transmit a notification indicating that the user is experiencing the symptoms of the medical scenario.

2. The ear-wearable device of claim 1, wherein the ML model comprises a decision tree model.

3. The ear-wearable device of claim 1, wherein the medical scenario comprises at least one of:
a medical overdose;
a medical substance withdrawal; or
a medical substance addiction.

4. The ear-wearable device of claim 1, wherein the one or more physiological parameters comprises at least one of:
a respiration pattern of the user;
a speech pattern of the user;
instances of vomiting by the user; or
instances of choking by the user.

5. The ear-wearable device of claim 4, wherein the respiration pattern comprises one or more of:
a delay between an exhalation and an inhalation by the user; or
an intra-breath spacing between temporally adjacent breaths by the user.

6. The ear-wearable device of claim 1, wherein the one or more physiological parameters comprises at least one of:
a change in a respiration rate of the user;
a change in a respiration pattern of the user; or
a change in a speech pattern of the user.

7. The ear-wearable device of claim 1, wherein the processing circuitry is further configured to:
receive, via the communications circuitry, user input indicating a medicinal history of the user; and
select, based on the received user input, the one or more physiological parameters from a plurality of available physiological parameters, wherein the one or more physiological parameters are indicative of symptoms of the medical scenario from a medical substance listed in the medicinal history of the user.

8. The ear-wearable device of claim 7, wherein the medicinal history comprises:
a type of a medical substance used by the user within a period of time; and
a dosage of the medical substance used by the user.

9. The ear-wearable device of claim 7, further comprising a memory configured to store a plurality of ML models, and wherein the processing circuitry is configured to:
select, based on the medical substance listed in the medicinal history of the user, the ML model from the plurality of ML models,
wherein each ML model of the plurality of ML models is configured to determine a likelihood that the user is experiencing the medical scenario from a corresponding medical substance.

10. The ear-wearable device of claim 1, wherein to cause the communications circuitry to transmit the notification indicating that the user is experiencing the symptoms of the medical scenario, the processing circuitry is configured to:
receive, via the communications circuitry, sensor data confirming that the user is experiencing the symptoms of the medical scenario; and
cause the communications circuitry to transmit the notification based on receipt of the sensor data.

11. The ear-wearable device of claim 10, wherein the sensor data comprises one or more of:
a body temperature of the user;
a change in the body temperature of the user;
a posture of the user;
a change in the posture of the user;
a heart rate of the user; or
a change in the heart rate of the user.

12. The ear-wearable device of claim 1, wherein the sensor comprises one or more of an accelerometer, an inertial measurement unit (IMU), a microphone, a photoplethysmography (PPG) sensor, or an oximeter.

13. A system comprising:
an ear-wearable device configured to be worn in, on, or about an ear of a user, the ear-wearable device comprising:
a sensor configured to sense one or more physiological parameters of the user;
and
memory; and
a processing system configured to:
retrieve, from ear-wearable device, the one or more sensed physiological parameters;
retrieve, from the memory, a machine learning (ML) model, wherein the ML model is configured to be trained via a training set comprising a plurality of physiological parameter values and a corresponding plurality of symptoms of a medical scenario;
apply the ML mode to the one or more sensed physiological parameters to determine whether a likelihood that the user is experiencing or will experience the medical scenario; and
based on a determination that the user is experiencing symptoms of the medical scenario, transmit a notification to a computing device indicating that the likelihood that the user is experiencing or will experience the medical scenario.

14. The system of claim 13, wherein the medical scenario comprises at least one of:
a medical overdose;
a medical substance withdrawal; or
a medical substance addiction.

15. The system of claim 13, wherein the processing system is configured to:
receive, from the ML model, a value corresponding to the likelihood that the user will experience the medical scenario;
compare the received value against a threshold value stored in the memory, wherein the threshold value corresponds to a threshold likelihood that the user will experience the medical scenario; and
based on a determination that the received value satisfies the threshold value, transmit the notification to the computing device.

16. The system of claim 13, wherein the one or more physiological parameters comprises one or more of:
a respiration pattern of the user;
a speech pattern or the user;
instances of vomiting by the user; or
instances of choking by the user.

17. The system of claim 16, wherein the respiration pattern comprises one or more of:
a delay between an exhalation and an inhalation by the user; or
an intra-breath spacing between temporally adjacent breaths by the user.

18. The system of claim 13, wherein the sensor comprises one or more of an accelerometer, an inertial measurement unit (IMU), a microphone, a photoplethysmography (PPG) sensor, or an oximeter.

19. The system of claim 13, wherein the processing system is configured to:
receive a medicinal history of the user; and
select, based on the medicinal history, the one or more physiological parameters from the plurality of physiological parameters, wherein the one or more physiological parameters are indicative of an occurrence of a possible medical scenario from a medical substance listed in the medicinal history.

20. A method comprising:
sensing, via a sensor disposed within an ear-wearable device configured to be worn in, on, or about an ear of a user, one or more physiological parameters of the user;
applying, by processing circuitry of the ear-wearable device, a machine learning (ML) model to the one or more physiological parameters to determine a likelihood that the user will experience a medical scenario; and
transmitting, by the processing circuitry and via communications circuitry of the ear-wearable device, a notification indicating the likelihood that the user will experience the medical scenario.
